(19) United States
(12) Patent Application Publication
(10) Pub. No.: US 2023/0184786 A1
(43) Pub. Date: Jun. 15, 2023

(54) Methods of Detecting and Treating Immune Responses Associated with Viral Infection

(71) Applicant: Forsyth Dental Infirmary for Children, d/b/a The Forsyth Institute, Cambridge, MA (US)

(72) Inventors: Markus Hardt, Belmont, MA (US); Fabian Schulte, Cambridge, MA (US); Wenyuan Shi, Boston, MA (US)

(21) Appl. No.: 17/912,677

(22) PCT Filed: Mar. 19, 2021
(86) PCT No.: PCT/US21/23348

§ 371 Date: Sep. 19, 2022

Related U.S. Application Data
(60) Provisional application No. 62/992,853, filed on Mar. 20, 2020.
(60) Provisional application No. 63/088,390, filed on Oct. 6, 2020.

(51) Int. Cl. G01N 33/68

(52) U.S. Cl. CPC G01N 33/6893; G01N 33/6851; G01N 2410/02; G01N 2410/06; G01N 2800/7095

(57) ABSTRACT

Provided herein are, in various embodiments, methods of detecting an acute inflammatory response associated with a viral infection in a patient, methods of predicting a likelihood of developing an acute inflammatory response (e.g., an acute respiratory distress syndrome) in a patient having a viral infection, such as a SARS-CoV2 infection, and methods of preparing a bodily fluid sample that is useful for performing the disclosed methods. The present invention also provides methods of treating a patient having a viral infection with a therapy that inhibits acute inflammation, such as acute inflammation mediated by the kinin-kallikrein system and/or the renin-angiotensin system (RAS).

### RELATED APPLICATION(S)

This application claims the benefit of U.S. Provisional Application No. 62/992,853, filed on Mar. 20, 2020 and U.S. Provisional Application No. 63/088,390, filed on Oct. 6, 2020. The entire teachings of the above applications are incorporated herein by reference.

### GOVERNMENT SUPPORT

This invention was made with Government support under Grant No. R21 DE029003 awarded by the National Institutes of Health. The Government has certain rights in the invention.

### INCORPORATION BY REFERENCE OF MATERIAL IN ASCII TEXT FILE

This application incorporates by reference the Sequence Listing contained in the following ASCII text file being submitted concurrently herewith:
a) File name: 57911000002 SequenceListing.txt; created Mar. 19, 2021, 9 KB in size.

### BACKGROUND

Coronaviruses are important human and animal pathogens. Recently, SARS-CoV-2, a novel coronavirus, first caused a cluster of pneumonia cases (COVID-19) in Wuhan, China, which rapidly spread, resulting in a pandemic throughout the world.

An estimation of disease severity in a report from the Chinese Center for Disease Control and Prevention that included approximately 44,500 confirmed infections, identified four categories. Symptoms in about 81% of the patients are mild, with no or mild pneumonia. Symptoms in about 14% of the patients are severe, for example, with dyspnea, hypoxia, or >50% lung involvement on imaging within 24 to 48 hours. Symptoms in about 5% of the patients are critical, for example, with respiratory failure, shock, or multiorgan dysfunction. The overall case fatality rate was 2.3% and no deaths were reported among noncritical cases.

Determination of the severity of the immunological host-response to infection by the novel coronavirus (SARS-CoV2) will aid in triaging patients that are strong responders to the disease and who may require additional medical attention including oxygen supplementation. Accordingly, there is a need for early identification of patients having a high risk of developing an acute inflammatory response.

### SUMMARY

The present invention is based, in part, on the finding that acute inflammatory response mediated by the kallikrein-kinin system and/or renin-angiotensin system (RAS) occurs in the bodily fluids of humans, and can be detected by quantifying peptide components of those systems in one or more human bodily fluids.

Accordingly, the invention described herein generally relates to methods of detecting an acute inflammatory response in a bodily fluid sample (e.g., a saliva sample, a urine sample or a blood sample).

One aspect of the invention relates to a method of detecting an acute inflammatory response, comprising quantifying a peptide within the kallikrein-kinin system (KKS), the renin-angiotensin system (RAS), or a combination thereof, in a bodily fluid sample, wherein the level of the peptide in the bodily fluid sample is indicative of an acute inflammatory response, or a lack thereof. In certain embodiments, the bodily fluid sample is obtained from a subject infected with or suspected of being infected with SARS-CoV2. In certain embodiments, the bodily fluid sample is a plasma sample.

Another aspect of the invention relates to a method of preparing a bodily fluid sample that is useful for detecting an acute inflammatory response, comprising:
a) obtaining or having obtained a bodily fluid sample;
b) adding a protease inhibitor, a control peptide, a retention-time standard peptide, or a combination thereof to the bodily fluid sample to prepare a sample that is useful for detecting an acute inflammatory response; and
c) quantifying a peptide within the kallikrein-kinin system (KKS), the renin-angiotensin system (RAS), or a combination thereof, in the sample prepared in step b).

In certain embodiments, the bodily fluid sample is obtained from a subject infected with or suspected of being infected with SARS-CoV2. In certain embodiments, the bodily fluid sample is a plasma sample.

An additional aspect of the invention relates to a method of predicting a likelihood of developing an acute inflammatory response (e.g., an acute respiratory distress syndrome) in a patient having a viral infection, comprising quantifying a peptide within the kallikrein-kinin system (KKS), the renin-angiotensin system (RAS), or a combination thereof, in a bodily fluid sample from the patient, wherein the level of the one or more peptides in the bodily fluid sample is indicative of the likelihood of developing an acute inflammatory response (e.g., an acute respiratory distress syndrome) in the patient. In certain embodiments, the patient is infected with SARS-CoV2. In certain embodiments, the bodily fluid sample is a plasma sample.

A further aspect of the invention relates to a method of classifying a patient having a viral infection based on a predicted likelihood of developing an acute inflammatory response, comprising:
a) quantifying a peptide within the kallikrein-kinin system (KKS), the renin-angiotensin system (RAS), or a combination thereof, in a bodily fluid sample from the patient;
b) predicting the likelihood of developing an acute inflammatory response based on the level of the peptide in the bodily fluid sample; and
c) classifying the patient based on the predicted likelihood.

In certain embodiments, the patient is infected with SARS-CoV2. In certain embodiments, the bodily fluid sample is a plasma sample.

Another aspect of the invention relates to a method of treating a patient having a viral infection, comprising:
a) quantifying a peptide within the kallikrein-kinin system (KKS), the renin-angiotensin system (RAS), or a combination thereof, in a bodily fluid sample of the patient;
b) identifying the patient as being likely to develop an acute inflammatory response (e.g., an acute respiratory distress syndrome) based on the level of the peptide in the bodily fluid sample; and
c) administering a therapy to the patient to inhibit acute inflammation.

In certain embodiments, the patient is infected with SARS-CoV2. In certain embodiments, the bodily fluid sample is a plasma sample.

An additional aspect of the invention relates to a method of monitoring progression of an acute inflammatory response (e.g., an acute respiratory distress syndrome) in a patient having a viral infection, comprising:
a) quantifying a peptide within the kallikrein-kinin system (KKS), the renin-angiotensin system (RAS), or a combination thereof, in a bodily fluid sample from the patient at a first time point;
b) repeating step a) at a second time point;
c) comparing the levels of the peptide in the bodily fluid sample at the first and second time points; and
d) determining the progression of the acute respiratory distress syndrome in the patient based on a change, or a lack thereof, in the levels of the peptide in the bodily fluid sample at the first and second time points.

In certain embodiments, the patient is infected with SARS-CoV2. In certain embodiments, the bodily fluid sample is a plasma sample.

Another aspect of the invention relates to a method of stratifying a set of patients having a viral infection, comprising:
a) quantifying a peptide within the kallikrein-kinin system (KKS), the renin-angiotensin system (RAS), or a combination thereof, in bodily fluid samples from individual patients in the set, wherein the level of the peptide in a bodily fluid sample is indicative of the likelihood of developing an acute inflammatory response (e.g., an acute respiratory distress syndrome) in a patient; and
b) stratifying the set of patients for treatment according to the individual patients' levels of the peptide in the bodily fluid samples.

In certain embodiments, the set of patients are infected with SARS-CoV2. In certain embodiments, the bodily fluid samples are plasma samples.

A further aspect of the invention relates to a method of ranking an urgency for treatment in a set of patients having a viral infection, comprising:
a) quantifying a peptide within the kallikrein-kinin system (KKS), the renin-angiotensin system (RAS), or a combination thereof, in bodily fluid samples from individual patients in the set, wherein the level of the peptide in a bodily fluid sample is indicative of the likelihood of developing an acute inflammatory response (e.g., an acute respiratory distress syndrome) in a patient; and
b) ranking the urgency for treating an acute inflammatory response (e.g., an acute respiratory distress syndrome) in the set of patients according to the individual patients' levels of the peptide in the bodily fluid samples.

In certain embodiments, the set of patients are infected with SARS-CoV2. In certain embodiments, the bodily fluid samples are plasma samples.

Another aspect of the invention relates to a method of processing a bodily fluid sample for detection of peptides indicative of a likelihood of developing an acute inflammatory response (e.g., an acute respiratory distress syndrome), comprising:
a) receiving a bodily fluid sample from a customer, wherein the bodily fluid sample was obtained from a patient having or suspected of having a viral infection;
b) adding a protease inhibitor, a control peptide, a retention-time standard peptide, or a combination thereof to the bodily fluid sample to prepare a sample useful for detecting an acute inflammatory response (e.g., an acute respiratory distress syndrome) in a patient;
c) quantifying a peptide within the kallikrein-kinin system (KKS), the renin-angiotensin system (RAS), or a combination thereof, in the sample prepared in step b);
d) generating a report based on levels of the peptide in the bodily fluid sample; and
e) delivering the report to the customer.

In certain embodiments, the patient is infected with or suspected of being infected with SARS-CoV2. In certain embodiments, the bodily fluid sample is a plasma sample.

Another aspect of the invention relates to a method of providing information regarding a patient's likelihood of developing an acute inflammatory response (e.g., an acute respiratory distress syndrome), comprising:
a) receiving a bodily fluid sample from a customer, wherein the bodily fluid sample was obtained from a patient having or suspected of having a viral infection;
b) adding a protease inhibitor, a control peptide, a retention-time standard peptide, or a combination thereof to the bodily fluid sample to prepare a sample useful for detecting an acute inflammatory response (e.g., an acute respiratory distress syndrome) in a patient;
c) quantifying a peptide within the kallikrein-kinin system (KKS), the renin-angiotensin system (RAS), or a combination thereof, in the sample prepared in step b);
d) generating a report based on levels of the peptide in the bodily fluid sample; and
e) delivering the report to the customer.

In certain embodiments, the patient is infected with or suspected of being infected with SARS-CoV2. In certain embodiments, the bodily fluid sample is a saliva sample.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 Longitudinal analysis of salivary Ang I ([Ang[1-10]), Ang II (Ang[1-8]), Ang [1-9] and Ang [1-7] over the course of the development of Sjögren's syndrome-like disease in NOD mice (4, 8, 12, 16-week time points) compared to Balb/C and B6 control mice.

FIG. 2 Immunohistochemical (IHC) for AT1R and AT2R expression in submandibular glands of mice.

FIGS. 3A-3B Overview of the renin-angiotensin system (RAS) (FIG. 3A) and kallikrein-kinin system (KKS) (FIG. 3B) and their connection to the SARS-CoV-host receptor ACE2 and pro- and anti-inflammatory signaling pathways. CP, carboxypeptidase, AP, aminopeptidase; NEP, neprilysin; LMW-kinin, low molecular weight kinin, HMW-kinin, high molecular weight kinin.

FIG. 4 Normalized whole saliva protein abundance for angiotensinogen (Angt) and kininogen (Kng1) across a cohort of Sjögren's syndrome patients and healthy controls (n=42).

FIG. 5 Protein sequence coverage of the Angt precursor is consistent with a proteolytic release of Ang I peptide (red) in saliva.

FIG. 6 Schematic overview of the targeted (MRM) LC-MS/MS acquisition workflow, for the accurate detection of SARS-CoV2 related peptides from the renin-angiotensin system.

FIG. 7 is a graphical representation of LC-MS/MS Chromatogram of Des-Arg-BK, Ang I, Ang II, Ang (1-7), BK, Ang (1-9), Kallidin, and Ang III.

FIGS. 8A-8B show calibration curves of Ang I, Ang II, Ang II (1-7), Ang I (1-9), Ang III (2-8), Brad (1-10), Brad (1-19) and Des-Arg9 Brad.

FIG. 9 shows principal component analysis (PCA) of plasma and saliva samples from healthy subjects.

FIG. 10 shows DesArg9-Brad, Brad 1-9, Brad 1-10, Ang 1-7, Ang 1-9, Ang II 1-7, Ang II 1-8, and Ang I 1-10 levels in blood samples from healthy subjects.

FIG. 11 shows DesArg9-Brad, Brad 1-9, Brad 1-10, Ang 1-7, Ang 1-9, Ang II 1-7, Ang II 1-8, and Ang I 1-10 levels in saliva samples from healthy subjects.

FIGS. 12A-12B show a comparison of levels of peptides associated with ACE activity, ACE2 activity or a combination thereof, in two bodily fluid samples (plasma v. saliva).

FIGS. 13A-13C show that a subset of the targeted RAS and KKS peptides defines moderate and severe COVID-19 disease. FIG. 13A. Hierarchical cluster analysis revealed that patients who received supplemental oxygen (black, dark grey) and non-infected controls (light grey) clustered in two distinct groups. FIG. 13B. Principal component analysis showed a separation of infected SARS-CoV-2 cases from uninfected controls (light grey) based on disease severity (grey, mild; dark grey, moderate; black, severe). FIG. 13C shows that decreased salivary levels of Angiotensin I (Ang[1-10] and Angiotensin III (Ang[2-8]) and increased plasma levels of Kallidin (K-BK[1-9]) differentiated infected individuals from uninfected controls.

FIGS. 14A-14B show enzyme surrogate activity measurements. FIG. 14A shows enzyme surrogate activity measurements defined as product/substrate ratios for ACE (Ang[1-8]/Ang[1-10]) and ACE2 (Ang[1-9]/Ang[1-10]) were significantly increased in saliva samples of SARS-CoV-2-infected individuals and mainly driven by a decrease of Ang[1-10]) in COVID-19 saliva samples. FIG. 14B shows aminopeptidase M surrogate activity was significantly reduced in COVID-19 plasma samples mainly driven by increased amounts of kallidin in viral infected samples.

FIGS. 15A-15B show plasma kallidin levels. FIG. 15A shows plasma kallidin levels at the initial first visit were significantly increased in individuals with more severe COVID-19 and FIG. 15B shows that kallidin were significantly increased at the initial first visit in those who required oxygen supplementation (Y) vs those who did not (N) or served as non-infected controls (C).

### DETAILED DESCRIPTION

A description of example embodiments follows.

Several aspects of the invention are described below, with reference to examples for illustrative purposes only. It should be understood that numerous specific details, relationships, and methods are set forth to provide a full understanding of the invention. One having ordinary skill in the relevant art, however, will readily recognize that the invention can be practiced without one or more of the specific details or practiced with other methods, protocols, reagents, cell lines and animals. The present invention is not limited by the illustrated ordering of acts or events, as some acts may occur in different orders and/or concurrently with other acts or events. Furthermore, not all illustrated acts, steps or events are required to implement a methodology in accordance with the present invention. Many of the techniques and procedures described, or referenced herein, are well understood and commonly employed using conventional methodology by those skilled in the art.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

The terminology used herein is for the purpose of describing particular embodiments only and is not intended to be limiting.

As used herein, the indefinite articles “a,” “an” and “the” should be understood to include plural reference unless the context clearly indicates otherwise.

Throughout this specification and the claims which follow, unless the context requires otherwise, the word “comprise,” and variations such as “comprises” and “comprising,” will be understood to imply the inclusion of, e.g., a stated integer or step or group of integers or steps, but not the exclusion of any other integer or step or group of integer or step. When used herein, the term “comprising” can be substituted with the term “containing” or “including.”

As used herein, “consisting of” excludes any element, step, or ingredient not specified in the claim element. When used herein, “consisting essentially of” does not exclude materials or steps that do not materially affect the basic and novel characteristics of the claim. Any of the terms “comprising,” “containing,” “including,” and “having,” whenever used herein in the context of an aspect or embodiment of the invention, can in some embodiments, be replaced with the term “consisting of,” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

The terms “protein,” “peptide” and “polypeptide” are used interchangeably herein to denote a polymer of at least two amino acids covalently linked by an amide bond, regardless of length or post-translational modification (e.g., glycosylation or phosphorylation). A protein, peptide or polypeptide can comprise any suitable L- and/or D-amino acid, for example, common α-amino acids (e.g., alanine, glycine, valine), non-α-amino acids (e.g., β-alanine, 4-aminobutyric acid, 6-aminocaproic acid, sarcosine, statine), and unusual amino acids (e.g., citrulline, homocitruline, homoserine, norleucine, norvaline, ornithine). The amino, carboxyl and/or other functional groups on a peptide can be free (e.g., unmodified) or protected with a suitable protecting group. Suitable protecting groups for amino and carboxyl groups, and methods for adding or removing protecting groups are known in the art and are disclosed in, for example, Green and Wuts, “Protecting Groups in Organic Synthesis,” John Wiley and Sons, 1991. The functional groups of a protein, peptide or polypeptide can also be derivatized (e.g., alkylated) or labeled (e.g., with a detectable label, such as a fluorogen or a hapten) using methods known in the art. A protein, peptide or polypeptide can comprise one or more modifications (e.g., amino acid linkers, acylation, acetylation, amidation, methylation, terminal modifiers (e.g., cyclizing modifications), N-methyl-α-amino group substitution), if desired. In addition, a protein, peptide or polypeptide can be an analog of a known and/or naturally-occurring peptide, for example, a peptide analog having conservative amino acid residue substitution(s).

The present invention provides, in various embodiments, methods of detecting and/or treating an acute inflammatory response.

In one aspect, the present invention provides a method of detecting an acute inflammatory response, comprising quantifying one or more peptides within the kallikrein-kinin system (KKS), the renin-angiotensin system (RAS), or a combination thereof, in a bodily fluid sample, wherein the level of the one or more peptides in the bodily fluid sample is indicative of an acute inflammatory response, or a lack thereof.

In another aspect, the present invention provides a method of detecting an acute inflammatory response, comprising quantifying a peptide within the kallikrein-kinin system (KKS) in a bodily fluid sample, wherein the level of the peptide in the bodily fluid sample is indicative of an acute inflammatory response, or a lack thereof.

In another aspect, the present invention provides a method of detecting an acute inflammatory response, comprising quantifying a peptide within the renin-angiotensin system (RAS) in a bodily fluid sample, wherein the level of the peptide in the bodily fluid sample is indicative of an acute inflammatory response, or a lack thereof.

As used herein, “the kallikrein-kinin system” or “the KKS” refers to the blood and tissue-based proteolytic pathways that generate kinin peptides (including bradykinin and kallidin) that are implicated in many physiological and pathological pathways including but not limited to the regulation of blood pressure and inflammation.

Non-limiting examples of peptides within the kallikrein-kinin system (KKS) include Kininogen, high molecular weight kinin (HMW-kinin), Bradykinin (BK[1-9]), des-Arg9-bradykinin (BK[1-8]), BK[1-7], BK[1-5], low molecular weight kinin (LMW-kinin), Kallidin (K-BK[1-9]), des-Arg10-kallidin (K-BK[1-8]) and K-BK[1-7].

In some embodiments, the acute inflammatory response is associated with an increase or a reduction of an enzyme within the kallikrein-kinin system (KKS). Non-limiting examples of enzymes within the kallikrein-kinin system include kallikrein (e.g., plasma kallikrein or tissue kallikrein), carboxypeptidase M (CPM), angiotensin-converting enzyme (ACE), angiotensin-converting enzyme 2 (ACE2), and aminopeptidase M (APM).

As used herein, “the renin-angiotensin system” or “the RAS” refers to blood and tissue-based proteolytic pathways that generate angiotensin peptides (including Angiotensin II) that are implicated in many physiological and pathological pathways including but not limited to the regulation of blood pressure and inflammation.

Non-limiting examples of peptides within the renin-angiotensin system (RAS) include Angiotensinogen (ANGT), Angiotensin (1-9) (Ang[1-9]), Angiotensin-(1-7) (Ang[1-7]), Angiotensin-(1-5) (Ang[1-5]), Angiotensin I (Ang[1-10]), Angiotensin II (Ang II, Ang[1-8]), Angiotensin-(2-10) (Ang-[2-10]), Angiotensin III (Ang III, Ang[2-8]), Angiotensin IV (Ang IV, Ang[3-8]) and Angiotensin-(3-7) (Ang[3-7]).

In some embodiments, the acute inflammatory response is associated with an increase or a reduction of an enzyme within the renin-angiotensin system (RAS). Non-limiting examples of enzymes within the renin-angiotensin system include Renin, angiotensin-converting enzyme 2 (ACE2), angiotensin-converting enzyme (ACE), neprilysin (NEP), aminopeptidase A (APA-A), aminopeptidase N (APA-N), and carboxypeptidase P (Cbp).

In some embodiments, the bodily fluid sample is from a subject. In some embodiments, the subject is a mammal. In some embodiments, the mammal is a patient (e.g., a human patient). In some embodiments, the patient is infected with a Severe Acute Respiratory Syndrome-related Coronavirus (SARS-CoV) or a Middle East Respiratory Syndrome-related Coronavirus (MERS-CoV). In some embodiments, the patient is infected with a SARS-CoV. In some embodiments, the patient is infected with a SARS-CoV2. In some embodiments, the patient has been diagnosed with COVID-19.

Non-limiting examples of bodily fluids include blood (e.g., whole blood and derivatives and fractions of blood, such as plasma or serum), bone marrow aspirates, cerebrospinal fluid, extracted galls, GCF gingival crevicular fluid, milk, prostate fluid, pus, saliva (including whole saliva, individual gland secretions, oral rinse), skin scrapes, sputum, surface washings, tears, urine, etc. In some embodiments, the bodily fluid comprises, consists essentially of or consists of blood, saliva, sputum, tears, urine or a combination thereof. In some embodiments, the bodily fluid comprises, consists essentially of or consists of plasma. In some embodiments, the bodily fluid comprises, consists essentially of or consists of saliva. In some embodiments, the bodily fluid comprises, consists essentially of or consists of plasma and saliva.

In some embodiments, the method further comprises preparing the bodily fluid sample by adding a protease inhibitor, a control peptide, a retention-time standard peptide, or a combination thereof.

In some embodiments, the protease inhibitor targets Cathepsin B, ACE2, Thimit oligopeptidase, MMP8, Neprilysin, ECE-1, ECE-2, Neprilysin-2, Chymase, Neutrophil elastase, tissue kallikrein, plasma kallikrein, aminopeptidase P, aminopeptidase M or a combination thereof. Non-limiting examples of protease inhibitors include leupeptin, E64d, antipain, DX600, MLN-4760, EDTA, RXP03, phosphoramidon, candoxatril, SM-19712, S136492, Chymostatin, alpha-1-antichymotrypsin, Sivelestat, ACE-inhibitors, etc.

In some embodiments, the control peptide is a stable-isotope labeled analog of the one or more peptides within the kallikrein-kinin system (KKS), the renin-angiotensin system (RAS), or a combination thereof. Non-limiting examples of control peptides include ClearPoint™ Angiotensin I (Ang[1-10]*, human, ¹³C and ¹⁵N labeled (AnaSpec): DR-V*-Y-I*-HPFHL (SEQ ID NO:1) [Val*=Val(U—¹³C^{5,15}N); Ile*=Ile(U—¹³C^{6,15}N)], ClearPoint™ Angiotensin II (Ang[1-8]*), human, ¹³C and ¹⁵N labeled (AnaSpec): DR-V*-Y-I*-HPF (SEQ ID NO:2) [Val*=Val(U—¹³C^{5,15}N); Ile*=Ile(U—¹³C^{6,15}N)] and Phe⁸Ψ(CH—NH)-Arg⁹-bradykinin (Tocris) or other stable isotope labeled or cleavage resistant analogs of KKS and RAS peptides.

In some embodiments, the retention-time standard peptide is selected from the group consisting of: LGGNETQVR (SEQ ID NO:3), AGGSSEPVTGLADK (SEQ ID NO:4), VEATFGVDESANK (SEQ ID NO:5), YILAGVESNK (SEQ ID NO:6), TPVISGGPYYER (SEQ ID NO:7), TPVITGAPYYER (SEQ ID NO:8), GDLDAASYYAPVR (SEQ ID NO:9), DAVTPADFSEWSK (SEQ ID NO:10), TGFIIDPGGVIR (SEQ ID NO:11), GTFIIDPAAIVR (SEQ ID NO:12), FLLQFGAQGSPLFK (SEQ ID NO:13) and combinations thereof.

In some embodiments, the method further comprises preparing the bodily fluid sample by enriching the sample for the one or more peptides within the kallikrein-kinin system (KKS), the renin-angiotensin system (RAS), or a combination thereof. In some embodiments, the peptide within the kallikrein-kinin system (KKS) or the renin-angiotensin system (RAS) is enriched by an immunoaffinity technique (e.g., using a monoclonal or polyclonal antibody specifically binds the peptide within the kallikrein-kinin system (KKS) or the renin-angiotensin system (RAS)).

In some embodiments, the peptide within the kallikrein-kinin system (KKS) and/or the renin-angiotensin system (RAS) in the bodily fluid sample is quantified using a mass-spectrometry-based assay, an antibody-based assay, or both. In some embodiments, the peptide within the kallikrein-kinin system (KKS) and/or the renin-angiotensin system (RAS) in the bodily fluid sample is quantified using a mass-spectrometry-based assay. In some embodiments, the mass-spectrometry-based assay is immuno-Matrix Assisted Laser Desorption/Ionization (iMALDI). In some embodiments, the iMALDI is a multiplexed iMALDI (e.g., duplex, triplex, quadruplex, pentaplex, or hexaplex).

In some embodiments, one peptide within the kallikrein-kinin system (KKS) in the bodily fluid sample is quantified. In some embodiments, two or more (e.g., 2, 3, 4, 5, 6, 7, 8, 9 or 10, or more) peptides within the kallikrein-kinin system (KKS) in the bodily fluid sample are quantified.

In some embodiments, the peptide within the kallikrein-kinin system (KKS) is selected from the group consisting of Kallidin (K-BK[1-9]), Bradykinin, [Des-Arg9]-Bradykinin, Lys-[Des-Arg9]-Bradykinin, RPPGFSP (SEQ ID NO:14), KRPPGFSP (SEQ ID NO:15), RPPGF (SEQ ID NO:22), KRPPGF (SEQ ID NO:23) and combinations thereof.

In some embodiments, the peptide within the kallikrein-kinin system (KKS) is Kallidin (K-BK[1-9]). In some embodiments, an above-threshold level of Kallidin in a bodily fluid sample (e.g., a bodily fluid sample associated with a systemic KKS response) is indicative of a higher likelihood for the patient to develop an acute inflammatory response. In some embodiments, an above-threshold level of Kallidin in a plasma sample is indicative of a higher likelihood for the patient to develop an acute inflammatory response.

In some embodiments, the threshold level of Kallidin is at least about 100 μg per mL of plasma sample, e.g., at least about 150 pg/mL, 175 pg/mL, 200 pg/mL, 225 pg/mL, 250 pg/mL, 275 pg/mL, 300 pg/mL, 310 pg/mL, 320 pg/mL, 330 pg/mL, 340 pg/mL or 350 pg/mL. In some embodiments, the threshold level of Kallidin is about 350 pg per mL of plasma sample, e.g., about: 150 pg/mL, 175 pg/mL, 200 pg/mL, 225 pg/mL, 250 pg/mL, 275 pg/mL, 300 pg/mL, 310 pg/mL, 320 pg/mL, 330 pg/mL, 340 pg/mL, 350 pg/mL, 360 pg/mL, 370 pg/mL, 380 pg/mL, 390 pg/mL, 400 pg/mL, 425 pg/mL, 450 pg/mL, 475 pg/mL, 500 pg/mL, 525 pg/mL, 550 pg/mL, 575 pg/mL, 600 pg/mL, 650 pg/mL, 700 pg/mL, 750 pg/mL, 800 pg/mL, 900 pg/mL or 1,000 pg/mL. In some embodiments, the threshold level of Kallidin is about 350 pg/mL.

In some embodiments:
a) an above-threshold level of Kallidin, Bradykinin, [Des-Arg9]-Bradykinin, Lys-[Des-Arg9]-Bradykinin, or a combination thereof;
b) a below-threshold level of RPPGFSP (SEQ ID NO:14), KRPPGFSP (SEQ ID NO:15), or both;
c) or a combination thereof,
   is indicative of an acute inflammatory response.

In some embodiments, the method further comprises determining a ratio of the levels of two different peptides within the kallikrein-kinin system (KKS).

In some embodiments, a below-threshold ratio of RPPGFSP (SEQ ID NO:14) Bradykinin, RPPGFSP (SEQ ID NO:14)/[Des-Arg9]-Bradykinin, KRPPGFSP (SEQ ID NO:15)/Lys-[Des-Arg9]-Bradykinin, KRPPGFSP (SEQ ID NO:15)/Kallidin or a combination thereof, is indicative of an acute inflammatory response.

In some embodiments, two or more (e.g., 2, 3, 4, 5, 6, 7, 8, 9 or 10, or more) peptides within the kallikrein-kinin system (KKS) in two or more bodily fluid samples are quantified. In some embodiments, the method further comprises determining a ratio of the ratios of the levels of two peptides within the kallikrein-kinin system (KKS) in two bodily fluid samples. In some embodiments, the ratio of ratios of RPPGFSP (SEQ ID NO:14)/[Des-Arg9]-Bradykinin, KRPPGFSP (SEQ ID NO:15)/Lys-[Des-Arg9]-Bradykinin, KRPPGFSP (SEQ ID NO:15)/Kallidin or a combination thereof, in a bodily fluid sample comprising a local KKS system response and a bodily fluid sample comprising a systemic KKS system response is determined.

Non-limiting examples of bodily fluid samples associated with a local KKS response include bone marrow aspirates, cerebrospinal fluid, extracted galls, GCF gingival crevicular fluid, milk, prostate fluid, pus, saliva (including whole saliva, individual gland secretions, oral rinse), skin scrapes, sputum, surface washings, tears and urine. Non-limiting examples of bodily fluid samples associated with a systemic KKS response include blood (e.g., whole blood or a derivative or fraction thereof, e.g., plasma or serum).

In some embodiments, an above-threshold ratio of ratios of two peptides within the kallikrein-kinin system (KKS), in a bodily fluid sample comprising a local KKS versus a bodily fluid sample comprising a systemic KKS, is indicative of an acute inflammatory response.

In some embodiments, an above-threshold ratio of two ratios, comprising 1) a ratio of levels of two different peptides within the kallikrein-kinin system (KKS) in a bodily fluid sample indicative of a local KKS response and 2) a ratio of levels of the same two peptides in a bodily fluid sample indicative of a systemic KKS response, is indicative of an acute inflammatory response. In some embodiments, a below-threshold ratio of the two ratios is indicative of an acute inflammatory response. In some embodiments, an above-threshold ratio of the two ratios is indicative of an acute inflammatory response.

In some embodiments, aminopeptidase M (APM) deactivation and/or downregulation associated with a systemic KKS response (e.g., in the plasma) is determined. In particular embodiments, the bodily fluid used to detect the systemic KKS response is blood. In some embodiments, the blood sample is whole blood. In other embodiments, the blood sample comprises a derivative or fraction of blood. In some embodiments, the blood sample comprises plasma, serum or a combination thereof. In other embodiments, the bodily fluid is urine.

In some embodiments, the method further comprises quantifying a peptide within the renin-angiotensin system (RAS) in a bodily fluid sample (e.g., a bodily fluid sample associated with a local RAS response such as saliva). In some embodiments, the method further comprises determining a ratio of a level of a peptide within the kallikrein-kinin system (KKS) (e.g. plasma Kallidin (K-BK[1-9])) and a level of a peptide within the renin-angiotensin system (RAS). In some embodiments, the peptide within the renin-angiotensin system (RAS) in the bodily fluid sample is quantified using a mass-spectrometry-based assay, an antibody-based assay, or both. In some embodiments, the peptide within the renin-angiotensin system (RAS) in the bodily fluid sample is quantified using a mass-spectrometry-based assay.

In some embodiments, the method further comprises preparing the bodily fluid sample by enriching the sample for the peptide within the renin-angiotensin system (RAS).

In some embodiments, one peptide within the renin-angiotensin system (RAS) in the bodily fluid sample is quantified. In some embodiments, two or more (e.g., 2, 3, 4, 5, 6, 7, 8, 9 or 10, or more) peptides within the renin-angiotensin system (RAS) in the bodily fluid sample are quantified.

In some embodiments, the peptide within the renin-angiotensin system (RAS) is selected from the group consisting of Angiotensin I (Ang I), Angiotensin III (Ang[2-8]), Angiotensinogen (ANGT), Angiotensin (1-9) (Ang-(1-9)), Angiotensin II (Ang II), Angiotensin-(1-7) (Ang-(1-7)), and combinations thereof.

In some embodiments, the peptide within the renin-angiotensin system (RAS) is Angiotensin I (Ang I). In other embodiments, the peptide within the renin-angiotensin system (RAS) is Angiotensin III (Ang[2-8]). In other embodiments, the peptide within the renin-angiotensin system (RAS) comprises both Angiotensin I (Ang I) and Angiotensin III (Ang[2-8]).

In some embodiments, a below-threshold level of Angiotensin I (Ang I) in a bodily fluid sample (e.g., a bodily fluid sample associated with a local RAS response) is indicative of a higher likelihood for the patient to develop an acute inflammatory response. In some embodiments, a below-threshold level of Angiotensin I (Ang I) in saliva is indicative of a higher likelihood for the patient to develop an acute inflammatory response.

In some embodiments, the threshold level of Angiotensin I (Ang I) is no more than about 100 μg per mL of saliva sample, e.g., no more than about: 95 pg/mL, 90 pg/mL, 89 pg/mL, 88 pg/mL, 87 pg/mL, 86 pg/mL, 85 pg/mL, 84 pg/mL, 83 pg/mL, 82 pg/mL, 81 pg/mL or 80 pg/mL. In some embodiments, the threshold level of Angiotensin I (Ang I) is about 80 pg per mL of saliva sample, e.g., about: 65 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 95 pg/mL or 100 pg/mL. In some embodiments, the threshold level of Angiotensin I (Ang I) is about 80 pg/mL.

In some embodiments, a below-threshold level of Angiotensin III (Ang[2-8]) in a bodily fluid sample (e.g., a bodily fluid sample associated with a local RAS response) is indicative of a higher likelihood for the patient to develop an acute inflammatory response. In some embodiments, a below-threshold level of Angiotensin III (Ang[2-8]) in saliva is indicative of a higher likelihood for the patient to develop an acute inflammatory response.

In some embodiments, the threshold level of Angiotensin III (Ang[2-8]) is no more than about 1,600 μg per mL of saliva sample, e.g., no more than about: 1,550 pg/mL, 1,500 pg/mL, 1,450 pg/mL, 1,400 pg/mL, 1,350 pg/mL, 1,340 pg/mL, 1,330 pg/mL, 1,320 pg/mL, 1,310 pg/mL or 1,300 pg/mL. In some embodiments, the threshold level of Angiotensin III (Ang[2-8]) is about 1,300 μg per mL of saliva sample, e.g., about 1,000 pg/mL, 1,050 pg/mL, 1,100 pg/mL, 1,150 pg/mL, 1,200 pg/mL, 1,200 pg/mL, 1,250 pg/mL, 1,260 pg/mL, 1,270 pg/mL, 1,280 pg/mL, 1,290 pg/mL, 1,300 pg/mL, 1,310 pg/mL, 1,320 pg/mL, 1,330 pg/mL, 1,340 pg/mL, 1,350 pg/mL, 1,400 pg/mL, 1,450 pg/mL, 1,500 pg/mL, 1,550 pg/mL or 1,600 pg/mL. In some embodiments, the threshold level of Angiotensin III (Ang[2-8]) is about 1,300 pg/mL.

In some embodiments:
a) a below-threshold level of Angiotensin I (Ang I) in saliva;
b) a below-threshold level of Angiotensin III (Ang[2-8]) in saliva; or
c) both a) and b),
   is indicative of a higher likelihood for the patient to develop an acute inflammatory response.

In some embodiments, an above-threshold level of Kallidin in a plasma sample, and:
a) a below-threshold level of Angiotensin I (Ang I) in a saliva sample;
b) a below-threshold level of Angiotensin III (Ang[2-8]) in a saliva sample; or
c) both a) and b),
   are indicative of a higher likelihood for the patient to develop an acute inflammatory response.

In some embodiments:
a) a below-threshold level of Ang I, Ang-(1-9), Ang-(1-7), or a combination thereof;
b) an above-threshold level of ANGT, Ang II, or both; or
c) a combination thereof,
   is indicative of an acute inflammatory response.

In some embodiments, the method further comprises determining a ratio of the levels of two different peptides within the renin-angiotensin system (RAS).

A ratio of the quantified levels of any two peptides within the renin-angiotensin system (RAS) can then be generated for a given bodily fluid sample (e.g., a saliva sample). Examples of useful ratios of levels of peptides within the renin-angiotensin system (RAS) include ratios of Ang II/Ang I, Ang-(1-7)/Ang-(1-9), Ang-(1-9)/Ang I, and Ang-(1-7)/Ang II. In some embodiments, the ratio of Ang II/Ang I is used to indicate ACE activity. In some embodiments, the ratio of Ang [1-7]/Ang II is used to indicate ACE2 activity.

In some embodiments, the ratio is indicative of the likelihood of the subject developing an acute inflammatory response. For example, in some embodiments, a below-threshold ratio of Ang-(1-7)/Ang I (e.g., a ratio of 0.1 or lower), Ang-II/Ang I (e.g., a ratio of 0.6 or lower, for example, 0.6, 0.5, 0.4, 0.3, 0.2, 0.1, 0.4-0.5, 0.4-0.6 or 0.5-0.6), Ang-(1-9)/Ang I (e.g., a ratio of 0.5 or lower), or a combination thereof, is indicative of an acute inflammatory response, or a likelihood of developing an acute inflammatory response.

In some embodiments, an above-threshold ratio of Ang II/Ang I, Ang-(1-7)/Ang-(1-9), or a combination thereof; a below-threshold ratio of Ang-(1-9)/Ang I, Ang-(1-7)/Ang II, or a combination thereof; or a combination thereof, is indicative of an acute inflammatory response, or a likelihood of developing an acute inflammatory response.

In some embodiments, renin or renin-like deactivation and/or downregulation associated with a local RAS response (e.g., in the oral cavity) is determined. In certain embodiments, the bodily fluid used to detect the local response is selected from the group consisting of bone marrow aspirates, cerebrospinal fluid, extracted galls, GCF gingival crevicular fluid, milk, prostate fluid, pus, saliva (including whole saliva, individual gland secretions, oral rinse), skin scrapes, sputum, surface washings, tears, urine and combinations thereof. In some embodiments, the saliva sample is stimulated whole saliva. In other embodiments, the saliva sample is unstimulated whole saliva.

In some embodiments, ACE2 deactivation and/or downregulation associated with a local RAS response is determined. In certain embodiments, the bodily fluid used to detect the local response is selected from the group consisting of bone marrow aspirates, cerebrospinal fluid, extracted galls, GCF gingival crevicular fluid, milk, prostate fluid, pus, saliva (including whole saliva, individual gland secretions, oral rinse), skin scrapes, sputum, surface washings, tears, urine and combinations thereof.

Low Ang II levels and low ACE activity (inferred by Ang I/Ang II ratios or measured with specific cleavage assay) are associated with increased mortality in severe sepsis (Zhang et al., Exp Ther Med. 7(5): 1342-48 (2014)) and vasodilatory shock (Bellomo, R. et al., Critical Care 24(1): 43 (2020) and Rice, C. et al., Circ. Shock 11(1): 59-63 (1983)). ACE activity decreases early in ARDS (Orfanos, S. et al., Circulation 102(16): 2011-18 (2000) and Reddy R et al., PLoS ONE 14(3): e0213096 (2019)). ARDS survivors have Ang(1-9)/Ang I ratio of 0.5 or higher (representing ACE2 activity), AngII/Ang I ratio of 0.4 or higher (representing ACE activity), and Ang(1-7)/Ang I ratio of 0.1 or higher (representing combined ACE and ACE2 or overall RAS activity) (Reddy R et al., PLoS ONE 14(3): e0213096 (2019)). Higher Ang I levels were associated with increased mortality, and higher Ang (1-9) levels were associated with decreased mortality (Reddy R et al., PLoS ONE 14(3): e0213096 (2019)). Trajectory after initial diagnosis is likely to be informative as well.

Accordingly, in some embodiments, ACE2 deactivation and/or downregulation associated with delocalized, or systemic, RAS response is determined. In particular embodiments, the bodily fluid used to detect the delocalized response is blood. In some embodiments, the blood sample is whole blood. In other embodiments, the blood sample comprises a derivative or fraction of blood. In some embodiments, the blood sample comprises plasma, serum or a combination thereof. In other embodiments, the bodily fluid is urine.

In particular embodiments, two or more (e.g., 2, 3, 4, 5, 6, 7, 8, 9 or 10, or more) peptides within the renin-angiotensin system (RAS) are quantified in at least two bodily fluids from a subject, wherein at least one bodily fluid sample is associated with a local RAS system response and at least one bodily fluid is associated with a de-localized (e.g., systemic) renin-angiotensin system (RAS) response. Non-limiting examples of bodily fluid samples associated with a local response include bone marrow aspirates, cerebrospinal fluid, extracted galls, GCF gingival crevicular fluid, milk, prostate fluid, pus, saliva (including whole saliva, individual gland secretions, oral rinse), skin scrapes, sputum, surface washings, tears and urine. Non-limiting examples of bodily fluid samples associated with a systemic response include blood (e.g., whole blood or a derivative or fraction thereof, e.g., plasma or serum).

Accordingly, in some embodiments of the methods disclosed herein, a ratio of two peptides within the renin-angiotensin system (RAS) is determined for at least two bodily fluid samples of a subject. In some embodiments, the ratio is a ratio of Ang II/Ang I, Ang-(1-7)/Ang I, Ang-(1-9)/Ang I, Ang-(1-7)/Ang-(1-9) or Ang II/Ang-(1-7). In some embodiments, the at least two bodily fluids include saliva and blood (e.g., serum). In some embodiments, the at least two bodily fluids include urine and blood (e.g., serum).

In some embodiments, an above-threshold ratio of two ratios, comprising 1) a ratio of levels of two different peptides within the renin-angiotensin system (RAS) in a bodily fluid sample indicative of a local RAS system response and 2) a ratio of levels of the same two peptides in a bodily fluid sample indicative of a systemic RAS system response, is indicative of an acute inflammatory response. In some embodiments, a below-threshold ratio of the two ratios is indicative of an acute inflammatory response. In some embodiments, an above-threshold ratio of the two ratios is indicative of an acute inflammatory response.

The bodily fluid sample, protease inhibitor, control peptide, retention-time standard peptide; the step of quantifying the peptide within the RAS system and the step of enriching the peptide within the RAS system in the sample are described herein.

In some embodiments, the acute inflammatory response comprises an acute respiratory distress syndrome (e.g., a severe respiratory distress syndrome), an acute kidney injury, an organ failure, or a combination thereof.

In some embodiments, the methods disclosed herein further comprise determining a genetic risk factor associated with the subject's likelihood of developing an acute inflammatory response upon becoming infected (e.g., with a SARS-Co-V2 virus), comprising detecting a genetic variant (e.g., via sequencing, PCR or hybridization) associated with an increased risk of developing and/or an increased severity of an acute inflammatory response. In some embodiments, the genetic variant associated with an increased infection risk and/or severity of an acute inflammatory response occurs in a gene selected from the group consisting of ACE, ACE2, AGT, Apolipoprotein E (Apo E), AT1R, aminopeptidase P (APP), kallikrein, Mannose-Biding Lectin, CD147, CCL2, Interleukin-12, and a human leukocyte antigen (HLA) class II gene, a HLA class III gene and combinations thereof. Methods of detecting genetic risk factor(s) (e.g., the genetic variant(s)) associated with an increased infection risk and/or severity of an acute inflammatory response are disclosed in, e.g., Gürkan, A. et al., Archives of oral biology 54(4): 337-44 (2009), Liu, C. et al., Cardiovascular Diabetology 17(1): 127 (2018), Rigat, B. et al., Journal of Clinical Investigation 86(4): 1343-46 (1990), Gürkan, A. et al., Journal of Clinical Periodontology 36(3): 204-11 (2009), Abel, H. et al., Mapping and characterization of structural variation in 17,795 human genomes, Nature (2020), Woodard-Grice et al., Pharmacogenetics and Genomics 20, 532-36 (2010), Gainer et al., Journal of Allergy and Clinical Immunology. 98(2):283-7 (1996), the contents of which are incorporated herein by reference.

In another aspect, the present invention provides a method of preparing a bodily fluid sample that is useful for detecting an acute inflammatory response, comprising:
a) obtaining or having obtained a bodily fluid sample;
b) adding a protease inhibitor, a control peptide, a retention-time standard peptide, or a combination thereof to the bodily fluid sample to prepare a sample that is useful for detecting an acute inflammatory response; and
c) quantifying one or more peptides within the kallikrein-kinin system (KKS), the renin-angiotensin system (RAS), or a combination thereof, in the sample prepared in step b).

In another aspect, the present invention provides a method of preparing a bodily fluid sample that is useful for detecting an acute inflammatory response, comprising:
a) obtaining or having obtained a bodily fluid sample;
b) adding a protease inhibitor, a control peptide, a retention-time standard peptide, or a combination thereof to the bodily fluid sample to prepare a sample that is useful for detecting an acute inflammatory response; and
c) quantifying a peptide within the kallikrein-kinin system (KKS) in the sample prepared in step b).

In another aspect, the present invention provides a method of preparing a bodily fluid sample that is useful for detecting an acute inflammatory response, comprising:
a) obtaining or having obtained a bodily fluid sample;
b) adding a protease inhibitor, a control peptide, a retention-time standard peptide, or a combination thereof to the bodily fluid sample to prepare a sample that is useful for detecting an acute inflammatory response; and
c) quantifying a peptide within the renin-angiotensin system (RAS) in the sample prepared in step b).

In some embodiments, the acute respiratory distress syndrome is a severe acute respiratory distress syndrome.

In some embodiments, the method further comprises enriching the peptide within the kallikrein-kinin system (KKS), the renin-angiotensin system (RAS), or a combination thereof, in the sample prior to quantifying in step c).

The bodily fluid sample, protease inhibitor, control peptide, retention-time standard peptide; the step of quantifying the peptide within the kallikrein-kinin system (KKS), the renin-angiotensin system (RAS), or a combination thereof, and the step of enriching the peptide within the kallikrein-kinin system (KKS), the renin-angiotensin system (RAS), or a combination thereof, in the sample are described herein.

In another aspect, the present invention provides a method of predicting a likelihood of developing an acute inflammatory response in a patient having a viral infection, comprising quantifying one or more peptides within the kallikrein-kinin system (KKS), the renin-angiotensin system (RAS), or a combination thereof, in a bodily fluid sample from the patient, wherein the level of the one or more peptides in the bodily fluid sample is indicative of the likelihood of developing an acute inflammatory response in the patient.

In another aspect, the present invention provides a method of predicting a likelihood of developing an acute inflammatory response (e.g., an acute respiratory distress syndrome) in a subject (e.g., a patient having a viral infection), comprising quantifying a peptide within the kallikrein-kinin system (KKS) in a bodily fluid sample from the subject, wherein the level of the peptide in the bodily fluid sample is indicative of the likelihood of developing an acute inflammatory response (e.g., an acute respiratory distress syndrome) in the subject.

In another aspect, the present invention provides a method of predicting a likelihood of developing an acute inflammatory response (e.g., an acute respiratory distress syndrome) in a subject (e.g., a patient having a viral infection), comprising quantifying a peptide within the renin-angiotensin system (RAS) in a bodily fluid sample from the subject, wherein the level of the peptide in the bodily fluid sample is indicative of the likelihood of developing an acute inflammatory response (e.g., an acute respiratory distress syndrome) in the subject.

In some embodiments, the subject is a mammal. In some embodiments, the mammal is a patient (e.g., a human patient). In some embodiments, the patient is infected with a Severe Acute Respiratory Syndrome-related Coronavirus (SARS-CoV) or a Middle East Respiratory Syndrome-related Coronavirus (MERS-CoV). In some embodiments, the patient is infected with a SARS-CoV. In some embodiments, the patient is infected with a SARS-CoV2. In some embodiments, the patient has been diagnosed with COVID-19.

The bodily fluid sample, protease inhibitor, control peptide, retention-time standard peptide; the step of quantifying the peptide within the kallikrein-kinin system (KKS), the renin-angiotensin system (RAS), or a combination thereof, and the step of enriching the peptide within the kallikrein-kinin system (KKS), the renin-angiotensin system (RAS), or a combination thereof, in the sample are described herein.

In some embodiments, an above-threshold level of Kallidin in plasma is indicative of a higher likelihood for the patient to develop an acute inflammatory response (e.g., an acute respiratory distress syndrome).

In some embodiments,
a) a below-threshold level of Angiotensin I (Ang I) in saliva;
b) a below-threshold level of Angiotensin III (Ang[2-8]) in saliva; or
c) both a) and b),
   is indicative of a higher likelihood for the patient to develop an acute inflammatory response (e.g., an acute respiratory distress syndrome).

In some embodiments, an above-threshold level of Kallidin in plasma and
a) a below-threshold level of Angiotensin I (Ang I) in saliva;
b) a below-threshold level of Angiotensin III (Ang[2-8]) in saliva; or
c) both a) and b),
   are indicative of a higher likelihood for the patient to develop an acute inflammatory response (e.g., an acute respiratory distress syndrome).

In some embodiments:
a) a below-threshold level of Ang I, Ang-(1-9), Ang-(1-7), or a combination thereof;
b) an above-threshold level of ANGT, Ang II, or both; or
c) a combination thereof,
   is indicative of the subject being likely to develop an acute inflammatory response (e.g., an acute respiratory distress syndrome).

In some embodiments:
a) an Ang II/ANGT ratio of about 1;
b) an above-threshold ratio of Ang II/Ang I, Ang-(1-7)/Ang-(1-9), or a combination thereof;
c) a below-threshold ratio of Ang-(1-9)/Ang I, Ang-(1-7)/Ang II, or a combination thereof; or
d) a combination thereof,
   is indicative of the subject being likely to develop an acute inflammatory response (e.g., an acute respiratory distress syndrome).

In some embodiments:
a) an above-threshold ratio of Ang II/Ang I, Ang-(1-7)/Ang-(1-9), or a combination thereof;
b) a below-threshold ratio of Ang-(1-9)/Ang I, Ang-(1-7)/Ang II, or a combination thereof; or
c) a combination thereof,
   is indicative of the subject being likely to develop an acute inflammatory response (e.g., an acute respiratory distress syndrome).

In some embodiments:
a) an above-threshold level of Bradykinin, [Des-Arg9]-Bradykinin, Lys-[Des-Arg9]-Bradykinin, Kallidin or a combination thereof;
b) a below-threshold level of RPPGFSP (SEQ ID NO:14), KRPPGFSP (SEQ ID NO:15), or both;
c) or a combination thereof,
   is indicative of the subject being likely to develop an acute inflammatory response (e.g., an acute respiratory distress syndrome).

In some embodiments, a below-threshold ratio of RPPGFSP (SEQ ID NO:14) Bradykinin, RPPGFSP (SEQ ID NO:14)/[Des-Arg9]-Bradykinin, KRPPGFSP (SEQ ID NO:15)/Lys-[Des-Arg9]-Bradykinin, KRPPGFSP (SEQ ID NO:15)/Kallidin or a combination thereof, is indicative of the subject being likely to develop an acute inflammatory response (e.g., an acute respiratory distress syndrome).

In some embodiments:
a) a below-threshold level of Ang I, Ang-(1-9), Ang-(1-7), or a combination thereof;
b) an above-threshold level of ANGT, Ang II, or both; or
c) a combination thereof,
   is indicative of the subject being likely to develop a severe acute respiratory distress syndrome.

In some embodiments:
a) an Ang II/ANGT ratio of about 1;
b) an above-threshold ratio of Ang II/Ang I, Ang-(1-7)/Ang-(1-9), or a combination thereof;
c) a below-threshold ratio of Ang-(1-9)/Ang I, Ang-(1-7)/Ang II, or a combination thereof; or
d) a combination thereof,
   is indicative of the subject being likely to develop a severe acute respiratory distress syndrome.

In some embodiments:
a) an above-threshold ratio of Ang II/Ang I, Ang-(1-7)/Ang-(1-9), or a combination thereof;
b) a below-threshold ratio of Ang-(1-9)/Ang I, Ang-(1-7)/Ang II, or a combination thereof; or
c) a combination thereof,
   is indicative of the subject being likely to develop a severe acute respiratory distress syndrome.

In some embodiments:
a) an above-threshold level of Bradykinin, [Des-Arg9]-Bradykinin, Lys-[Des-Arg9]-Bradykinin, Kallidin or a combination thereof;
b) a below-threshold level of RPPGFSP (SEQ ID NO:14), KRPPGFSP (SEQ ID NO:15), or both;
c) or a combination thereof,
   is indicative of the subject being likely to develop a severe acute respiratory distress syndrome.

In some embodiments, a below-threshold ratio of RPPGFSP (SEQ ID NO:14) Bradykinin, RPPGFSP (SEQ ID NO:14)/[Des-Arg9]-Bradykinin, KRPPGFSP (SEQ ID NO:15)/Lys-[Des-Arg9]-Bradykinin, KRPPGFSP (SEQ ID NO:15)/Kallidin or a combination thereof, is indicative of the subject being likely to develop a severe acute respiratory distress syndrome.

In another aspect, the present invention provides a method of classifying a patient having a viral infection based on a predicted likelihood of developing an acute inflammatory response, comprising:
a) quantifying one or more peptides within the kallikrein-kinin system (KKS), the renin-angiotensin system (RAS), or a combination thereof, in a bodily fluid sample from the patient;
b) predicting the likelihood of developing an acute inflammatory response based on the level of the one or more peptides in the bodily fluid sample; and
c) classifying the patient based on the predicted likelihood.

In another aspect, the present invention provides a method of classifying a patient having a viral infection based on a predicted likelihood of developing an acute inflammatory response (e.g., an acute respiratory distress syndrome), comprising:
a) quantifying a peptide within the kallikrein-kinin system (KKS) in a bodily fluid sample from the patient;
b) predicting the likelihood of developing an acute inflammatory response (e.g., an acute respiratory distress syndrome) based on the level of the peptide in the bodily fluid sample; and
c) classifying the patient based on the predicted likelihood.

In another aspect, the present invention provides a method of classifying a patient having a viral infection based on a predicted likelihood of developing an acute inflammatory response (e.g., an acute respiratory distress syndrome), comprising:
a) quantifying a peptide within the renin-angiotensin system (RAS) in a bodily fluid sample from the patient;
b) predicting the likelihood of developing an acute inflammatory response (e.g., an acute respiratory distress syndrome) based on the level of the peptide in the bodily fluid sample; and
c) classifying the patient based on the predicted likelihood.

In another aspect, the present invention provides a method of treating a patient having a viral infection, comprising:
a) quantifying one or more peptides within the kallikrein-kinin system (KKS), the renin-angiotensin system (RAS), or a combination thereof, in a bodily fluid sample of the patient;
b) identifying the patient as being likely to develop an acute inflammatory response based on the level of the one or more peptides in the bodily fluid sample; and
c) administering a therapy to the patient to inhibit acute inflammation.

In another aspect, the present invention provides a method of treating a patient having a viral infection, comprising:
a) quantifying a peptide within the kallikrein-kinin system (KKS) in a bodily fluid sample of the patient;
b) identifying the patient as being likely to develop an acute inflammatory response (e.g., an acute respiratory distress syndrome) based on the level of the peptide in the bodily fluid sample; and
c) administering a therapy to the patient to inhibit acute inflammation.

In another aspect, the present invention provides a method of treating a patient having a viral infection, comprising:
a) quantifying a peptide within the renin-angiotensin system (RAS) in a bodily fluid sample of the patient;
b) identifying the patient as being likely to develop an acute inflammatory response (e.g., an acute respiratory distress syndrome) based on the level of the peptide in the bodily fluid sample; and
c) administering a therapy to the patient to inhibit acute inflammation.

In some embodiments, the therapy comprises a therapeutically effective amount of:
a) an agent that inhibits B1R, B2R, or both B1R and B2R;
b) an agent that blocks tissue kallikrein activity, plasma kallikrein activity, or both;
c) an agent that blocks the production of kallidin, bradykinin, low molecular weight kinin, high molecular weight kinin, kininogen, BK[1-8] or K-BK[1-8], or a combination thereof; or
a combination of a)-c).

In some embodiments, the agent that inhibits B2R is a B2-receptor antagonist. In some embodiments, the B2-receptor antagonist is icatibant. See, e.g., Veerdonk F L van de et al., Jama Network Open 3:e2017708 (2020), for additional information on treating Patients With COVID-19 using icatibant, the contents of which is incorporated herein by reference in their entirety.

In some embodiments, the therapy comprises a therapeutically effective amount of an agent that inhibits AT1R, an agent that blocks ANG II production, or a combination thereof.

In some embodiments, the therapy comprises a therapeutically effective amount of an agent that inhibits AT1R. In some embodiments, the agent that inhibits AT1R includes a sartan, an Angiotensin II receptor blocker (ARB), or a combination thereof.

In some embodiments, the therapy comprises a therapeutically effective amount of an agent that blocks ANG II production. In some embodiments, the agent that blocks ANG II production includes an Angiotensin-converting-enzyme inhibitor (ACE inhibitor), a renin inhibitor, or a combination thereof.

In some embodiments, the therapy comprises a therapeutically effective amount of an agent that increases the expression and/or function of Alamandine receptor, AT1R, AT2R, AT4, Mas, MrgD receptor or a combination thereof. In some embodiments, the agent that increases AT1R expression and/or function includes Ang II, C-reactive protein, an Ang [1-7] analog (e.g., cyclic Ang(1-7) or NorLeu3 Ang(1-7)) or a combination thereof. (See, e.g., Zangrillo, A. et al., Critical Care 24(1): 227 (2020), Wang, C. et al., Circulation 107(13): 1783-90 (2003)).

In some embodiments, the therapy comprises a therapeutically effective amount of an agent selected from the group consisting of: an ACE inhibitor, an Angiotensin II type-1 receptor blocker, an Angiotensin II type-2 receptor agonist, a MAS receptor agonist, an ACE2 activator, an agent that blocks binding interface between SARS-CoV2 and ACE2, a soluble ACE2, an agent that blocks ANG II production, and combinations thereof. In some embodiments, the agent is selected from the group consisting of: Captopril, candesartan, Compound 21 (C21), AVE 0991, rhACE2, rhACE2, Anti-ACE2 antibody, and combinations thereof.

“Treating,” as used herein, refers to taking steps to deliver a therapy to a subject, such as a mammal (e.g., a human patient), in need thereof (e.g., as by administering to a mammal one or more therapeutic agents). “Treating” includes inhibiting the disease or condition (e.g., as by slowing or stopping its progression or causing regression of the disease or condition), and relieving the symptoms resulting from the disease or condition.

“A therapeutically effective amount” is an amount effective, at dosages and for periods of time necessary, to achieve a desired therapeutic result (e.g., treatment, healing, inhibition or amelioration of physiological response or condition, etc.). The full therapeutic effect does not necessarily occur by administration of one dose, and may occur only after administration of a series of doses. Thus, a therapeutically effective amount may be administered in one or more administrations. A therapeutically effective amount may vary according to factors such as disease state, age, sex, and weight of a mammal, mode of administration and the ability of a therapeutic, or combination of therapeutics, to elicit a desired response in an individual.

An effective amount of an agent to be administered can be determined by a clinician of ordinary skill using the guidance provided herein and other methods known in the art. For example, suitable dosages can be from about 0.001 mg/kg to about 100 mg/kg, from about 0.01 mg/kg to about 100 mg/kg, from about 0.01 mg/kg to about 10 mg/kg, from about 0.01 mg/kg to about 1 mg/kg body weight per treatment. Determining the dosage for a particular agent, subject and disease is well within the abilities of one of skill in the art. Preferably, the dosage does not cause or produces minimal adverse side effects.

A therapeutic agent described herein can be administered via a variety of routes of administration, including, for example, oral, dietary, topical, transdermal, rectal, parenteral (e.g., intra-arterial, intravenous, intramuscular, subcutaneous injection, intradermal injection), intravenous infusion and inhalation (e.g., intrabronchial, intranasal or oral inhalation, intranasal drops) routes of administration, depending on the compound and the particular disease to be treated. Administration can be local or systemic as indicated. The preferred mode of administration can vary depending on the particular compound chosen.

In another aspect, the present invention provides a method of monitoring progression of an acute inflammatory response in a patient having a viral infection, comprising:
a) quantifying one or more peptides within the kallikrein-kinin system (KKS), the renin-angiotensin system (RAS), or a combination thereof, in a bodily fluid sample from the patient at a first time point;
b) repeating step a) at a second time point;
c) comparing the levels of the one or more peptides in the bodily fluid sample at the first the second time points; and
d) determining the progression of the acute respiratory distress syndrome in the patient based on a change, or a lack thereof, in the levels of the one or more peptides in the bodily fluid sample at the first and second time points.

In another aspect, the present invention provides a method of monitoring progression of an acute inflammatory response (e.g., an acute respiratory distress syndrome) in a patient having a viral infection, comprising:
a) quantifying a peptide within the kallikrein-kinin system (KKS) in a bodily fluid sample from the patient at a first time point;
b) repeating step a) at a second time point;
c) comparing the levels of the peptide in the bodily fluid sample at the first the second time points; and
d) determining the progression of the acute respiratory distress syndrome in the patient based on a change, or a lack thereof, in the levels of the peptide in the bodily fluid sample at the first and second time points.

In another aspect, the present invention provides a method of monitoring progression of an acute inflammatory response (e.g., an acute respiratory distress syndrome) in a patient having a viral infection, comprising:
a) quantifying a peptide within the renin-angiotensin system (RAS) in a bodily fluid sample from the patient at a first time point;
b) repeating step a) at a second time point;
c) comparing the levels of the peptide in the bodily fluid sample at the first the second time points; and
d) determining the progression of the acute respiratory distress syndrome in the patient based on a change, or a lack thereof, in the levels of the peptide in the bodily fluid sample at the first and second time points.

In some embodiments, step a) is repeated twice or more (e.g., 3, 4, 5, 6, 7, or 8 times). In some embodiments, the determining a change, or a lack thereof, in the levels of the one or more peptides in the bodily fluid sample occurs between any of the time points.

In some embodiments, a therapy is administered to the patient between the first the second time points. In some embodiments, a therapy is administered to the patient between the first and the last time points.

In another aspect, the present invention provides a method of stratifying a set of patients having a viral infection, comprising:
a) quantifying one or more peptides within the kallikrein-kinin system (KKS), the renin-angiotensin system (RAS), or a combination thereof, in bodily fluid samples from individual patients in the set, wherein the level of the one or more peptides in a bodily fluid sample is indicative of the likelihood of developing an acute inflammatory response in a patient; and
b) stratifying the set of patients for treatment according to the individual patients' levels of the one or more peptides in the bodily fluid samples.

In another aspect, the present invention provides a method of stratifying a set of patients having a viral infection, comprising:
a) quantifying a peptide within the kallikrein-kinin system (KKS) in bodily fluid samples from individual patients in the set, wherein the level of the peptide in a bodily fluid sample is indicative of the likelihood of developing an acute inflammatory response (e.g., an acute respiratory distress syndrome) in a patient; and
b) stratifying the set of patients for treatment according to the individual patients' levels of the peptide in the bodily fluid samples.

In another aspect, the present invention provides a method of stratifying a set of patients having a viral infection, comprising:
a) quantifying a peptide within the renin-angiotensin system (RAS) in bodily fluid samples from individual patients in the set, wherein the level of the peptide in a bodily fluid sample is indicative of the likelihood of developing an acute inflammatory response (e.g., an acute respiratory distress syndrome) in a patient; and
b) stratifying the set of patients for treatment according to the individual patients' levels of the peptide in the bodily fluid samples.

In another aspect, the present invention provides a method of ranking an urgency for treatment in a set of patients having a viral infection, comprising:
a) quantifying one or more peptides within the kallikrein-kinin system (KKS), the renin-angiotensin system (RAS), or a combination thereof, in bodily fluid samples from individual patients in the set, wherein the level of the one or more peptides in a bodily fluid sample is indicative of the likelihood of developing an acute inflammatory response in a patient; and
b) ranking the urgency for treating an acute inflammatory response in the set of patients according to the individual patients' levels of the one or more peptides in the bodily fluid samples.

In another aspect, the present invention provides a method of ranking an urgency for treatment in a set of patients having a viral infection, comprising:
a) quantifying a peptide within the kallikrein-kinin system (KKS) in bodily fluid samples from individual patients in the set, wherein the level of the peptide in a bodily fluid sample is indicative of the likelihood of developing an acute inflammatory response (e.g., an acute respiratory distress syndrome) in a patient; and
b) ranking the urgency for treating an acute inflammatory response (e.g., an acute respiratory distress syndrome) in the set of patients according to the individual patients' levels of the peptide in the bodily fluid samples.

In another aspect, the present invention provides a method of ranking an urgency for treatment in a set of patients having a viral infection, comprising:
a) quantifying a peptide within the renin-angiotensin system (RAS) in bodily fluid samples from individual patients in the set, wherein the level of the peptide in a bodily fluid sample is indicative of the likelihood of developing an acute inflammatory response (e.g., an acute respiratory distress syndrome) in a patient; and
b) ranking the urgency for treating an acute inflammatory response (e.g., an acute respiratory distress syndrome) in the set of patients according to the individual patients' levels of the peptide in the bodily fluid samples.

In another aspect, the present invention provides a method of preparing a bodily fluid sample useful for predicting an acute inflammatory response (e.g., an acute respiratory distress syndrome) in a patient having a viral infection, comprising:
a) obtaining or having obtained the bodily fluid sample from the patient;
b) adding a protease inhibitor, a control peptide, a retention-time standard peptide, or a combination thereof to the bodily fluid sample to prepare a sample that is useful for predicting an acute inflammatory response (e.g., an acute respiratory distress syndrome) in a patient; and
c) quantifying one or more peptides within the kallikrein-kinin system (KKS), the renin-angiotensin system (RAS), or a combination thereof, in the sample prepared in step b).

In some embodiments, the method further comprises enriching the one or more peptides within the kallikrein-kinin system (KKS), the renin-angiotensin system (RAS), or a combination thereof, in the sample prior to quantifying in step c). In some embodiments, the peptide within the kallikrein-kinin system (KKS) or the renin-angiotensin system (RAS) is enriched based on immunoaffinity (e.g., using a monoclonal or polyclonal antibody specifically binds the peptide within the kallikrein-kinin system (KKS) or the renin-angiotensin system (RAS)).

In another aspect, the present invention provides a method of preparing a bodily fluid sample useful for predicting an acute inflammatory response (e.g., an acute respiratory distress syndrome) in a patient having a viral infection, comprising:
a) obtaining or having obtained the bodily fluid sample from the patient;
b) adding a protease inhibitor, a control peptide, a retention-time standard peptide, or a combination thereof to the bodily fluid sample to prepare a sample that is useful for predicting an acute inflammatory response (e.g., an acute respiratory distress syndrome) in a patient; and
c) quantifying a peptide within the kallikrein-kinin system (KKS) in the sample prepared in step b).

In some embodiments, the method further comprises enriching the peptide within the kallikrein-kinin system (KKS) in the sample prior to quantifying in step c). In some embodiments, the peptide within the kallikrein-kinin system (KKS) is enriched based on immunoaffinity (e.g., using a monoclonal or polyclonal antibody specifically binds the peptide within the kallikrein-kinin system (KKS)).

In another aspect, the present invention provides a method of preparing a bodily fluid sample useful for predicting an acute inflammatory response (e.g., an acute respiratory distress syndrome) in a patient having a viral infection, comprising:
a) obtaining or having obtained the bodily fluid sample from the patient;
b) adding a protease inhibitor, a control peptide, a retention-time standard peptide, or a combination thereof to the bodily fluid sample to prepare a sample that is useful for predicting an acute inflammatory response (e.g., an acute respiratory distress syndrome) in a patient; and
c) quantifying a peptide within the renin-angiotensin system (RAS) in the sample prepared in step b).

In some embodiments, the method further comprises enriching the peptide within the renin-angiotensin system (RAS) in the sample prior to quantifying in step c). In some embodiments, the peptide within the renin-angiotensin system (RAS) is enriched based on immunoaffinity (e.g., using a monoclonal or polyclonal antibody specifically binds the peptide within the renin-angiotensin system (RAS)).

In another aspect, the present invention provides a method of processing a bodily fluid sample for detection of peptides indicative of a likelihood of developing an acute inflammatory response, comprising:
a) receiving a bodily fluid sample from a customer, wherein the bodily fluid sample was obtained from a patient having or suspected of having a viral infection;
b) adding a protease inhibitor, a control peptide, a retention-time standard peptide, or a combination thereof to the bodily fluid sample to prepare a sample useful for detecting an acute inflammatory response in a patient;
c) quantifying one or more peptides within the kallikrein-kinin system (KKS), the renin-angiotensin system (RAS), or a combination thereof, in the sample prepared in step b);
d) generating a report based on levels of the one or more peptides in the bodily fluid sample; and
e) delivering the report to the customer.

In some embodiments, the method further comprises enriching the one or more peptides within the kallikrein-kinin system (KKS), the renin-angiotensin system (RAS), or a combination thereof, in the sample prior to quantifying in step c). In some embodiments, the peptide within the kallikrein-kinin system (KKS) or the renin-angiotensin system (RAS) is enriched based on immunoaffinity (e.g., using a monoclonal or polyclonal antibody specifically binds the peptide within the kallikrein-kinin system (KKS) or the renin-angiotensin system (RAS)).

In another aspect, the present invention provides a method of processing a bodily fluid sample for detection of peptides indicative of a likelihood of developing an acute inflammatory response (e.g., an acute respiratory distress syndrome), comprising:
a) receiving a bodily fluid sample from a customer, wherein the bodily fluid sample was obtained from a patient having or suspected of having a viral infection;
b) adding a protease inhibitor, a control peptide, a retention-time standard peptide, or a combination thereof to the bodily fluid sample to prepare a sample useful for detecting an acute inflammatory response (e.g., an acute respiratory distress syndrome) in a patient;
c) quantifying a peptide within the kallikrein-kinin system (KKS) in the sample prepared in step b);
d) generating a report based on levels of the peptide in the bodily fluid sample; and
e) delivering the report to the customer.

In some embodiments, the method further comprises enriching the peptide within the kallikrein-kinin system (KKS) in the sample prior to quantifying in step c). In some embodiments, the peptide within the kallikrein-kinin system (KKS) is enriched based on immunoaffinity (e.g., using a monoclonal or polyclonal antibody specifically binds the peptide within the kallikrein-kinin system (KKS)).

In another aspect, the present invention provides a method of processing a bodily fluid sample for detection of peptides indicative of a likelihood of developing an acute inflammatory response (e.g., an acute respiratory distress syndrome), comprising:
a) receiving a bodily fluid sample from a customer, wherein the bodily fluid sample was obtained from a patient having or suspected of having a viral infection;
b) adding a protease inhibitor, a control peptide, a retention-time standard peptide, or a combination thereof to the bodily fluid sample to prepare a sample useful for detecting an acute inflammatory response (e.g., an acute respiratory distress syndrome) in a patient;
c) quantifying a peptide within the renin-angiotensin system (RAS) in the sample prepared in step b);
d) generating a report based on levels of the peptide in the bodily fluid sample; and
e) delivering the report to the customer.

In some embodiments, the method further comprises enriching the peptide within the renin-angiotensin system (RAS) in the sample prior to quantifying in step c). In some embodiments, the peptide within the renin-angiotensin system (RAS) is enriched based on immunoaffinity (e.g., using a monoclonal or polyclonal antibody specifically binds the peptide within the renin-angiotensin system (RAS)).

In some embodiments, the customer is a hospital, a doctor's office, a medical research lab or facility, a government agency, or a combination thereof.

In another aspect, the present invention provides a method of providing information regarding a patient's likelihood of developing an acute inflammatory response, comprising:
a) receiving a bodily fluid sample from a customer, wherein the bodily fluid sample was obtained from a patient having or suspected of having a viral infection;
b) adding a protease inhibitor, a control peptide, a retention-time standard peptide, or a combination thereof to the bodily fluid sample to prepare a sample useful for detecting an acute inflammatory response in a patient;
c) quantifying one or more peptides within the kallikrein-kinin system (KKS), the renin-angiotensin system (RAS), or a combination thereof, in the sample prepared in step b);
d) generating a report based on levels of the one or more peptides in the bodily fluid sample; and
e) delivering the report to the customer.

In some embodiments, the method further comprises enriching the one or more peptides within the kallikrein-kinin system (KKS), the renin-angiotensin system (RAS), or a combination thereof, in the sample prior to quantifying in step c). In some embodiments, the peptide within the kallikrein-kinin system (KKS) or the renin-angiotensin system (RAS) is enriched based on immunoaffinity (e.g., using a monoclonal or polyclonal antibody specifically binds the peptide within the kallikrein-kinin system (KKS) or the renin-angiotensin system (RAS)).

In another aspect, the present invention provides a method of providing information regarding a patient's likelihood of developing an acute inflammatory response (e.g., an acute respiratory distress syndrome), comprising:
a) receiving a bodily fluid sample from a customer, wherein the bodily fluid sample was obtained from a patient having or suspected of having a viral infection;
b) adding a protease inhibitor, a control peptide, a retention-time standard peptide, or a combination thereof to the bodily fluid sample to prepare a sample useful for detecting an acute inflammatory response (e.g., an acute respiratory distress syndrome) in a patient;
c) quantifying a peptide within the kallikrein-kinin system (KKS) in the sample prepared in step b);
d) generating a report based on levels of the peptide in the bodily fluid sample; and
e) delivering the report to the customer.

In some embodiments, the method further comprises enriching the peptide within the kallikrein-kinin system (KKS) in the sample prior to quantifying in step c). In some embodiments, the peptide within the kallikrein-kinin system (KKS) is enriched based on immunoaffinity (e.g., using a monoclonal or polyclonal antibody specifically binds the peptide within the kallikrein-kinin system (KKS)).

In another aspect, the present invention provides a method of providing information regarding a patient's likelihood of developing an acute inflammatory response (e.g., an acute respiratory distress syndrome), comprising:
a) receiving a bodily fluid sample from a customer, wherein the bodily fluid sample was obtained from a patient having or suspected of having a viral infection;
b) adding a protease inhibitor, a control peptide, a retention-time standard peptide, or a combination thereof to the bodily fluid sample to prepare a sample useful for detecting an acute inflammatory response (e.g., an acute respiratory distress syndrome) in a patient;
c) quantifying a peptide within the renin-angiotensin system (RAS) in the sample prepared in step b);
d) generating a report based on levels of the peptide in the bodily fluid sample; and
e) delivering the report to the customer.

In some embodiments, the method further comprises enriching the peptide within the renin-angiotensin system (RAS) in the sample prior to quantifying in step c). In some embodiments, the peptide within the renin-angiotensin system (RAS) is enriched based on immunoaffinity (e.g., using a monoclonal or polyclonal antibody specifically binds the peptide within the renin-angiotensin system (RAS)).

In some embodiments, the method further comprises administering a therapy to inhibit acute inflammation in a patient identified as having a higher likelihood of developing an acute inflammatory response (e.g., an acute respiratory distress syndrome). In some embodiments, the acute respiratory distress syndrome is a severe acute respiratory distress syndrome. The therapy is any one of the therapies described herein.

In some embodiments, the method further comprises admitting a patient identified as having a higher likelihood of developing a severe acute respiratory distress syndrome for in-hospital treatment.

In some embodiments, the method further comprises triaging the patient based on the likelihood for the patient to develop an acute inflammatory response (e.g., an acute respiratory distress syndrome). In some embodiments, the acute respiratory distress syndrome is a severe acute respiratory distress syndrome.

### EXAMPLES

### Example 1. LC-MS/MS Assay of Bodily Fluid Samples

Sample Collection. Human whole bodily fluid was sampled by expectorating into a collection tube after letting bodily fluid pool at the bottom of the mouth. Bodily fluid samples were centrifuged at 12,000 g for 10 min at 4° C. to remove cells and the supernatants were aliquoted and stored at −80° C. within 4 h of collection.

Sample processing. Insoluble material was removed from each sample by centrifuging 500 μL of bodily fluid at 12,000 g for 5 min at 4° C. 450 μL of the supernatant were aspirated into an ultrafiltration device (Vivaspin VS0102, nominal molecular weight limit 10,000) and centrifuged at 12,000 g for 90 min at 4° C. The filtrate was acidified with TFA to a final concentration of 0.4% and desalted over monolithic C-18-tips (Pierce, Rockford, Ill.).

Liquid chromatography electrospray ionization tandem mass spectrometry (nanoLC-ESI-MS/MS). 5 μL of each sample with iRTh peptide standards was injected for data acquisition in randomized order. Individual and pooled samples were analyzed using an Orbitrap Fusion (q-OT-qIT) tribrid mass spectrometer (Thermo Scientific) equipped with an EASY-nLC 1000 ultra-high-pressure liquid chromatography unit (Thermo Scientific). Tryptic digests were loaded onto a trap column (Acclaim PepMap 100, 100 μm×2 cm) and subsequently separated on a 50 cm EASY-Spray column (E5803, 75 μm×50 cm, C18, 2 μm, 100 Å). Mobile phase buffer A was composed of 0.1% formic acid in water; mobile phase buffer B was composed of acetonitrile and 0.1% formic acid. Samples were loaded onto the trap column for 9 min at 2 μl/min. Mobile phase B increased from 2% to 32% at 150 min at a flowrate of 200 nL/min followed by a 30-min wash at 72% and a 20-min re-equilibration at 0% B. Data was acquired in positive ionization mode in data-dependent acquisition mode (DDA). DDA-acquisition parameters were as follows: Precursor ion survey scans from 350 to 1200 m/z were acquired at 60 k resolution (at 200 m/z) with an automatic gain control (AGC) target of 3.0×10⁶ ion counts. Subsequent MS/MS analyses were con-ducted using a top speed approach over a 3-sec cycle. The most abundant precursors (intensity greater than 2.0×10⁴ ions) were isolated in the quadrupole mass analyzer using a 2.5-Da m/z isolation window, and fragmented by higher-energy collisional dissociation (HCD) using a normalized collision energy of 30. Fragment ions were detected in the Orbitrap mass analyzer with a resolution setting of 15,000, an AGC target setting 2×10⁵ and a maximum ion accumulation time of 150 msec. Previously analyzed precursor ions were dynamically excluded for 80 s using a 10-ppm exclusion mass window. Only precursors with charge states between 2 and 8 were selected for MS/MS and monoisotopic precursor selection was turned on. Label-free quantitation was performed using the PEAKS Q module. Mass error tolerance was set to 10 ppm between runs and the retention time shift tolerance was set to 5 minutes. Reference and training samples were autodetected. Results on the peptide level were exported and processed using a custom R-script using the Bioconductor limma software package. Peak areas were normalized using the total ion current of samples.

### Example 2. Identification of a Local Bodily Fluid RAS

Severe acute respiratory syndrome-related coronavirus (SARS-CoV) first emerged in humans in 2003 after transmitting from palm civets in Guangzhou Province, China (Wang, M. et al., SARS-CoV Infection in a Restaurant from Palm, Civet. Emerg Infect Dis 11: 1860-65 (2005)). SARS-CoV and the newly emerging SARS-CoV2 (Wuhan Province, China) belong to lineage B of beta-coronaviruses. SARS-CoV-inoculated macaques excrete SARS-CoV from nose, mouth and pharynx two days after infection (Kuiken, T. et al., Newly discovered coronavirus as the primary cause of severe acute respiratory syndrome, Lancet 362: 263-70 (2003)). Cell entry is essential component of transmission. All CoVs encode a surface glycoprotein, spike, which binds to a host-cell receptor and mediates viral entry (Li, F. Structure, Function, and Evolution of Coronavirus Spike Proteins, Ann Rev Virol 3: 237-61 (2016)). Angiotensin-converting enzyme 2 (ACE2) is the host-cell receptor for lineage B CoVs (SARS-CoV and SARS-CoV2) (Kuba, K. et al., A crucial role of angiotensin converting enzyme 2 (ACE2) in SARS coronavirus-induced lung injury, Nat Med 11: 875-79 (2005); Wan, Y., Shang, J., Graham, R., Baric, R. S. & Li, F., Receptor recognition by novel coronavirus from Wu-han: An analysis based on decade-long structural studies of SARS, J Virol (2020)). Saliva of SARS-CoV infected individuals carry high viral loads (6×10⁸ copies/ml) (Wang, W.-K. et al., Detection of SARS-associated coronavirus in throat wash and saliva in early diagnosis, Emerg Infect Dis 10: 1213-19 (2004)). Likewise, the virus is consistently detected in saliva collected from SARS-CoV2-infected individuals (To, K. K.-W. et al., Consistent detection of 2019 novel coronavirus in saliva, Clin Infect Dis (2020)).

Viral replication in the oral cavity may significantly contribute to the rapid viral shedding into saliva droplets and disease transmission. Epithelial cells lining the salivary gland ducts are early target cells of SARS-CoV in rhesus macaques (Liu, L. et al., Epithelial cells lining salivary gland ducts are early target cells of severe acute respiratory syndrome coronavirus infection in the upper respiratory tracts of rhesus macaques, J Virol 85: 4025-30 (2011)). Epithelial cells of the oral mucosa have been shown to highly express ACE2, the host-receptor of SARS-CoV2 (Xu, H. et al., High expression of ACE2 receptor of 2019-nCoV on the epithelial cells of oral mucosa, Int J Oral Sci 12(8) (2020)). A local renin-angiotensin system has been previously detected in the parotid, sublingual and submandibular salivary glands of rats (Cano, I. P. et al., Losartan and isoproterenol promote alterations in the local renin-angiotensin system of rat salivary glands, PLoS ONE 14: e0217030 (2019)).

In the classical RAS, the main effector peptide Ang II is produced from angiotensinogen through sequential enzymatic cleavages catalyzed by renin and angiotensin-converting enzyme (ACE) (Reid, I. A., Morris, B. J. & Ganong, W. F., The renin-angiotensin system, Annual Review of Physiology 40: 377-410 (1978)). Local, tissue-based RAS (Paul, M., Wagner, J. & Dzau, V. J., Gene expression of the renin-angiotensin system in human tissues. Quantitative analysis by the polymerase chain reaction, Journal of Clinical Investigation 91: 2058-64 (1993)) can exert organ specific functions that independent of the circulatory RAS and that include regulation of cell growth, differentiation, proliferation, apoptosis and ROS generation (Gasparo, M. de, Catt, K. J., Inagami, T., Wright, J. W. & Unger, T., International union of pharmacology. XXIII. The angiotensin II receptors. Pharmacological reviews 52: 415-72 (2000)). Ang II signal via AT1R to promote the expression of pro-inflammatory mediators including cytokines, chemokines and adhesion molecules in both resident tissue and infiltrating immune cells (Nataraj, C. et al., Angiotensin II regulates cellular immune responses through a calcineurin-dependent path-way, Journal of Clinical Investigation 104: 1693-1701 (1999); Suzuki, Y. et al., Inflammation and angiotensin II, The international journal of biochemistry & cell biology 35: 881-900 (2003); Chaparro, R. J. et al., Nonobese diabetic mice express aspects of both type 1 and type 2 diabetes, Proceedings of the National Academy of Sciences of the United States of America 103: 12475-480 (2006)). RAS blocker (ACEi, ARBs) that reduce ATR1-mediated signaling can act as anti-inflammatory and immunomodulatory agents (Saitou, M. et al., Functional specialization of human salivary glands and origins of proteins intrinsic to human saliva, bioRxiv 96: 2020.02.12.945659 (2020)). ACEi and ARB medications are widely used in patients with cardiovascular disease and have excellent safety records in their long-term use without significant side effects (Imai, Y. et al., Angiotensin-converting enzyme 2 protects from severe acute lung failure, Nature 436: 112-16 (2005)). However, the extent of a salivary gland-based RAS in uncertain.

### Example 2A. Identification of a Local Salivary RAS in Mouse Salivary Glands

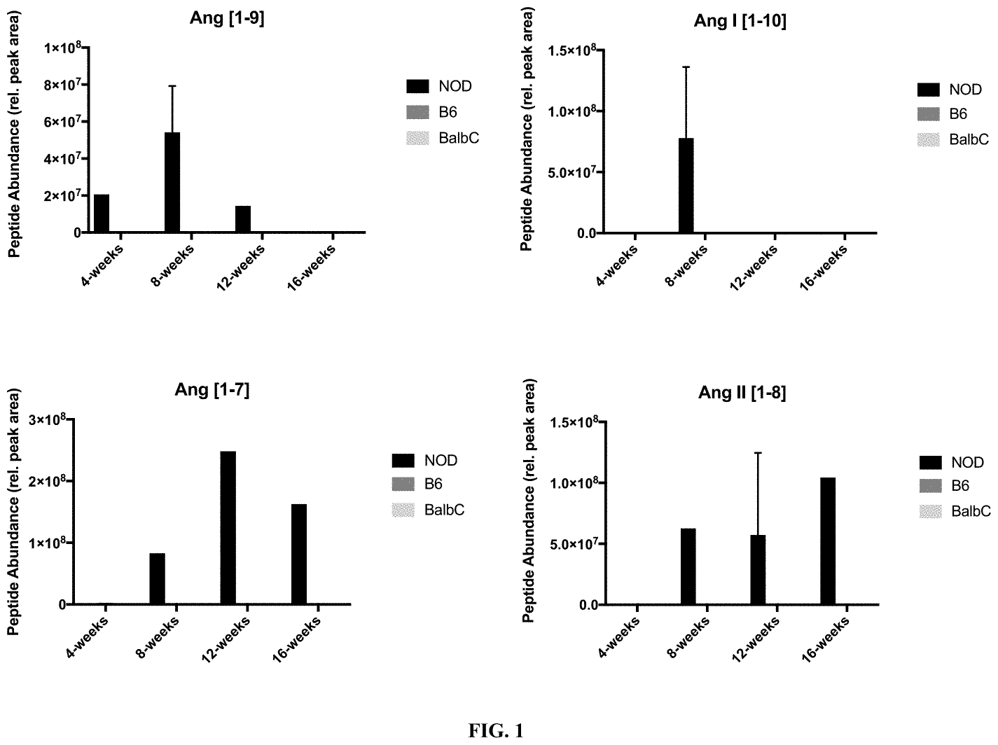

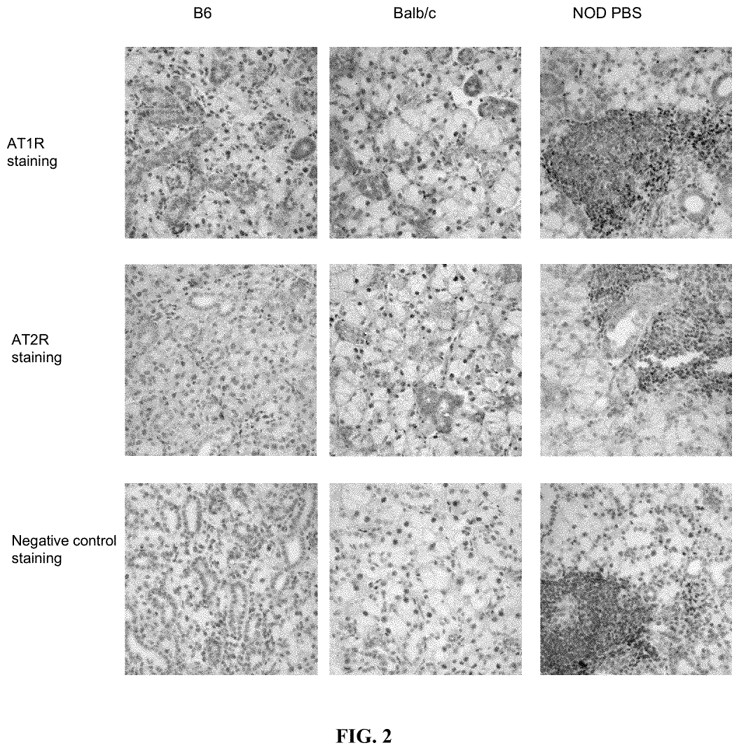

Non-obese diabetic mice (NOD) are a widely accepted model of SjS (Karnik, S. S. et al., International Union of Basic and Clinical Pharmacology. XCIX. Angiotensin Receptors: Interpreters of Pathophysiological Angiotensinergic Stimuli [corrected], Pharmacological reviews 67: 754-819 (2015)). NOD mice belong to mouse strains with intrinsic high levels of submandibular renin2, which catalyzes the initial, rate-limiting step in the RAS cascade, making them ideal to study salivary RAS (Gasparo, M. de, Catt, K. J., Inagami, T., Wright, J. W. & Unger, T., International union of pharmacology. XXIII. The angiotensin II receptors, Pharmacological reviews 52: 415-72 (2000)). Longitudinally, levels of Ang II and Ang [1-7] increased over the course of SjS-development (4, 8, 12 and 16-weeks) and were not detected in B6 and Balb/C control mice. (FIG. 1). AT1 and AT2 receptor were detected by immunohistochemistry (IHC) on ductal epithelial cells, acinar epithelial cells and some infiltrating immune cells in the leukocyte foci in submandibular glands of mice. As expected, B6 and Balb/c control mice did not have leukocyte foci in the submandibular glands. Presence of Ang [1-9] and Ang [1-7] peptides, as well as MAS receptor on murine salivary epithelial cells and infiltrating immune cells (IHC, RT-PCR) demonstrate the presence of counterbalancing, protective Ang [1-7]/Mas axis in addition to the classic RAS.

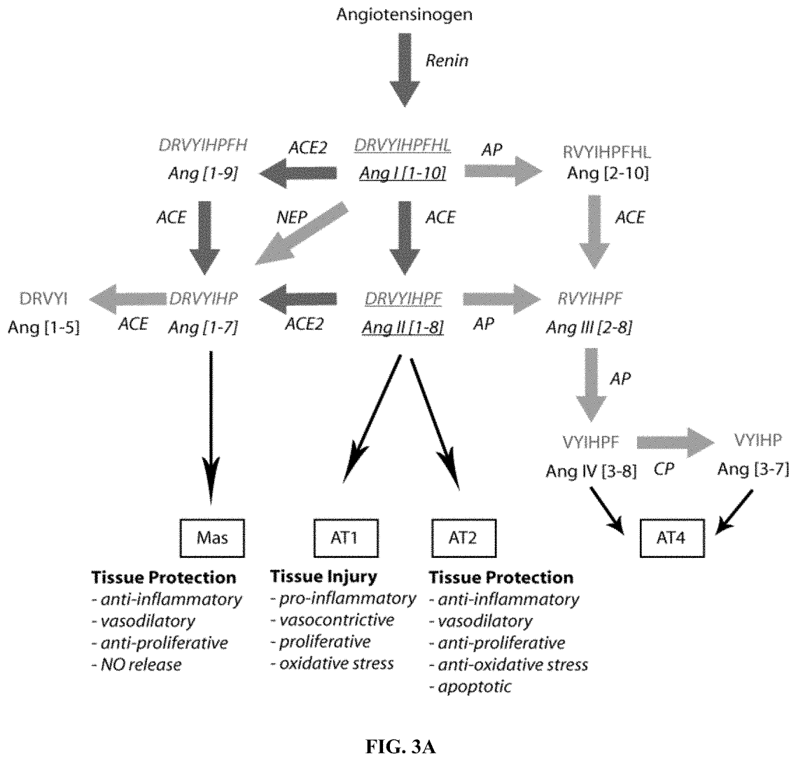

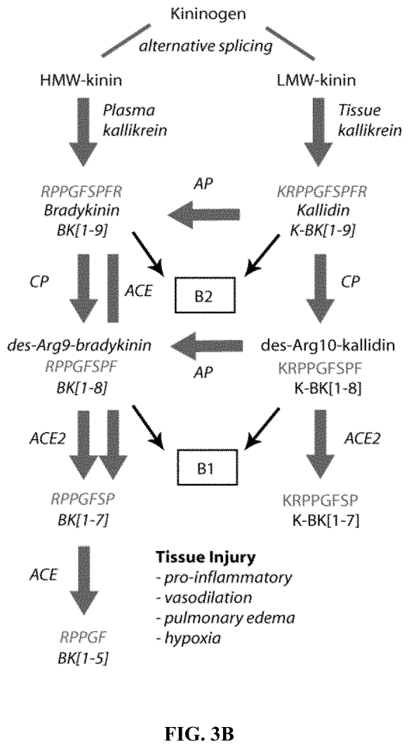

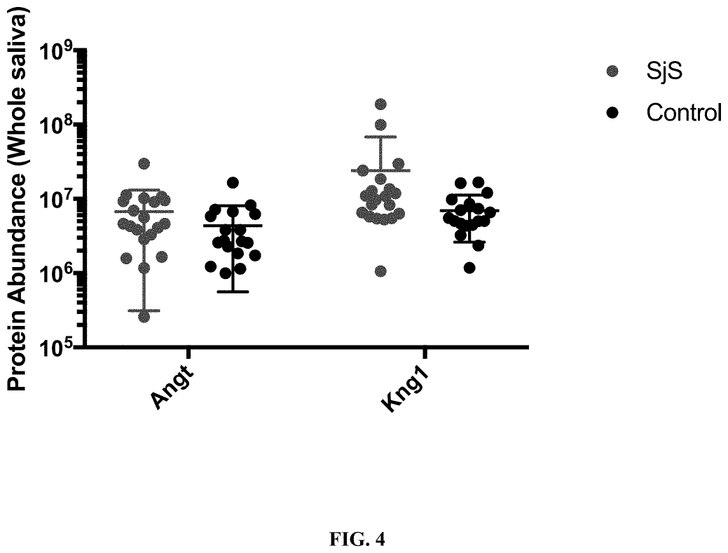

The present invention identifies a local salivary RAS. The salivary RAS, comprised of angiotensinogen (AGT), angiotensin-converting enzyme-1 and -2 (ACE1 and ACE2), the receptors AT1, AT2 and MAS and angiotensin peptides Ang I [1-10], Ang II [1-8] and the ACE2 products Ang [1-9] and Ang [1-7], was detected in NOD mice using LC-MS/MS peptidomics, immunohistochemistry and quantitative RT-PCR (FIG. 3). The detection of angiotensin peptides in saliva collected from NOD mice was favored by the high expression of renin in the salivary glands, which is typically the rate-limiting step of RAS.

### Example 2B. Identification of a Local Salivary RAS in Human Salivary Glands

In humans, angiotensinogen (AGT) and kininogen (KIN1), another precursor of a substrate (bradykinin) for ACE2) in saliva are detected by LC-MS/MS-based proteomics. Genes for all of the RAS components (angiotensinogen (AGT), renin (REN), angiotensin-converting enzyme-1 (ACE), angiotensin-converting enzyme-2 (ACE2), angiotensin II receptor type 1 (AGTR1), angiotensin II receptor type 2 (AGTR2) and MAS receptor (MAS 1)) as well as kininogen (KNG1) are transcribed in the major human salivary glands (parotid, submandibular, sublingual; Table 1) (Reid, I. A., Morris, B. J. & Ganong, W. F., The renin-angiotensin system, Annual Review of Physiology 40: 377-410 (1978)).

Certain RAS components, specifically the precursor proteins angiotensinogen, kininogen and renin receptor have been reported in published proteomics study of human saliva Grassl N, Kulak NA, Pichler G, Geyer PE, Jung J, et al., Ultra-deep and quantitative saliva proteome reveals dynamics of the oral microbiome, Genome medicine 8(1): 44 (2016); Hall S C, Hassis M E, Williams K E, Albertolle M E, Prakobphol A, et al., Alterations in the Salivary Proteome and N-Glycome of Sjögren's Syndrome Patients, Journal of Proteome Research 16(4): 1693-1705 (2017); Lin, Y.-H. et al., Self-Assembled STrap for Global Proteomics and Salivary Biomarker Discovery, J Proteome Res 18: 1907-15 (2019).

Using ultradeep shotgun sequencing entailing upfront prefractionation, Grassl et al. were also able to detect angiotensin-converting enzyme-1 (ACE), angiotensin-converting enzyme-2 (ACE2) that presumably were shed from the plasma membrane.

Angiotensin and kinin peptides are expected to be in much lower concentrations in human samples. Indeed, even in plasma, angiotensin peptides are of low abundance (˜19 amol/μL) and their quantitation routinely involves enzymatic incubation times to boost peptide levels. Therefore, it is not surprising that salivary angiotensin peptides have so far eluded routine LC-MS/MS peptidomics analyses.

By switching to targeted acquisition modes, both multiple-reaction monitoring (MRM; Bystrom C E, Salameh W, Reitz R, Clarke N J., Plasma renin activity by LC-MS/MS: development of a prototypical clinical assay reveals a subpopulation of human plasma samples with substantial peptidase activity, Clinical Chemistry 56(10):1561-69 (2010)) and immunoaffinity-MALDI (Mason D R, Reid J D, Camenzind A G, Holmes D T, Borchers C H., Duplexed iMALDI for the detection of angiotensin I and angiotensin II, Methods 56(2): 213-22 (2012) have been able to quantify Ang I and II from 50 μL plasma with a limit of detection of ˜10 amol/μL.

In salivary proteomics studies, Angt and Kng1 proteins have been consistently detected across donors with occasional missing data points. FIG. 3 shows the detected relative protein abundances of Angt and Kng1 in whole saliva across a cohort of Sjögren's syndrome (SjS) patients and healthy controls. Both Angt and Kng1 precursor protein were slightly more abundant in SjS patients mirroring previously reported results (Hall S C, Hassis M E, Williams K E, Albertolle M E, Prakobphol A, et al., Alterations in the Salivary Proteome and N-Glycome of Sjögren's Syndrome Patients, Journal of Proteome Research 16(4):1693-1705 (2017)). In companion salivary peptidomics studies, Lysyl-bradykinin has occasionally been identified. However, the employed discovery-based LC-MS/MS acquisition approaches lack the sensitivity for routine detection.

Gene expression analysis shows that all genes of the RAS pathway (ANGT, REN, ACE, ACE2, ANGTR1, ANGTR2 and MAS1 as well as KNG1) are transcribed in the major human salivary glands (Table; (Saitou et al. 2020)).

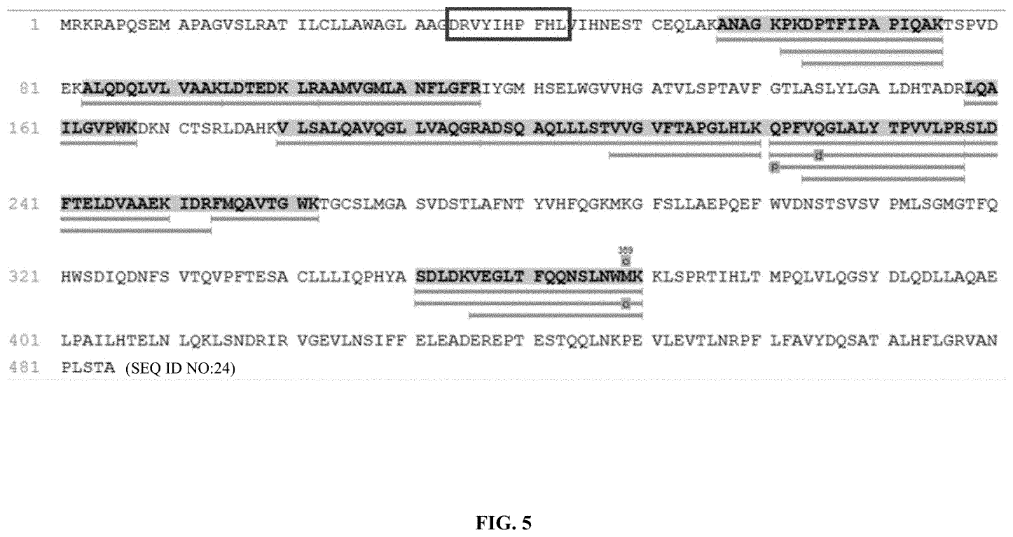

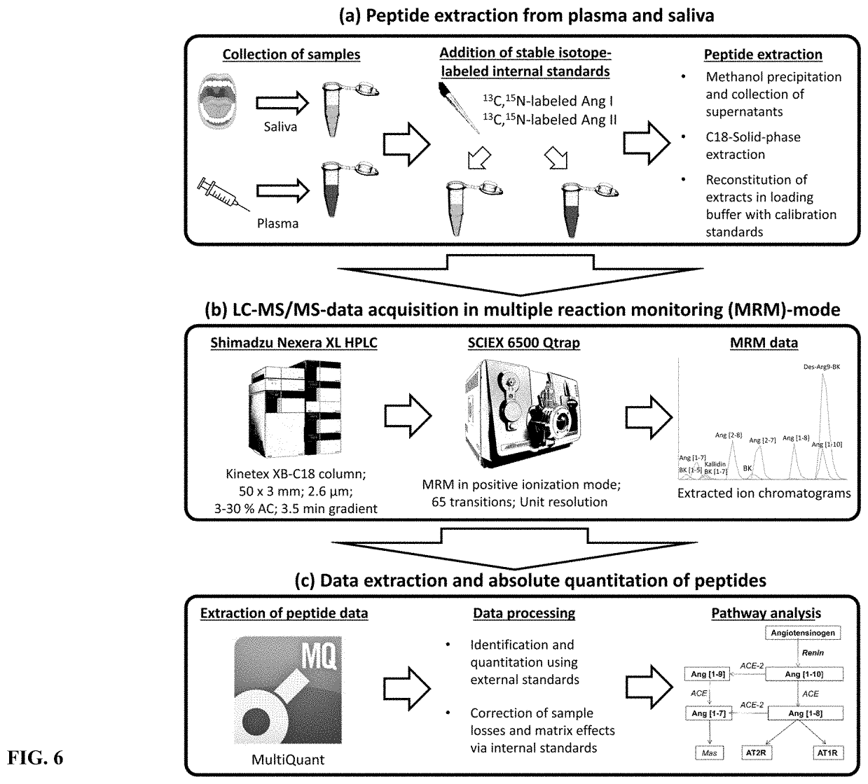

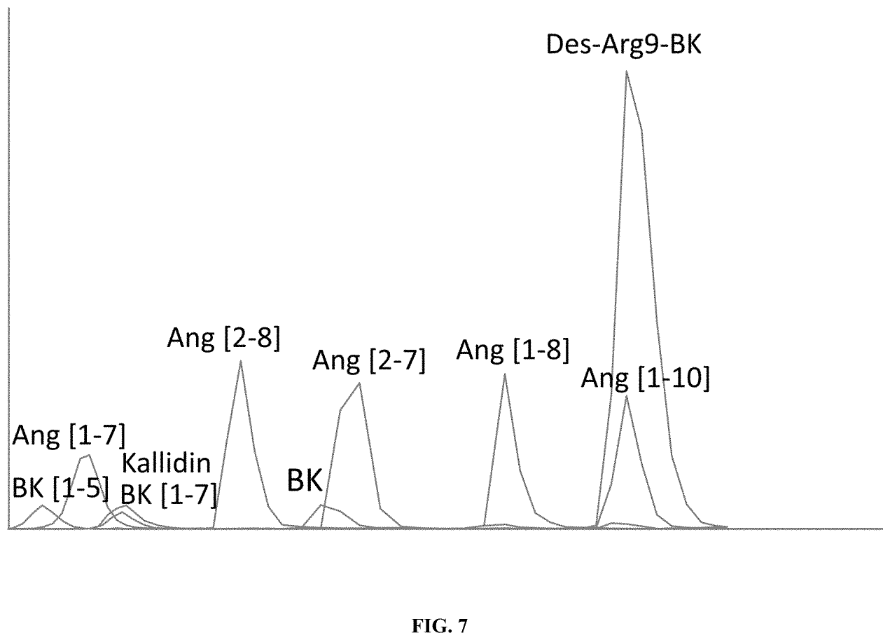

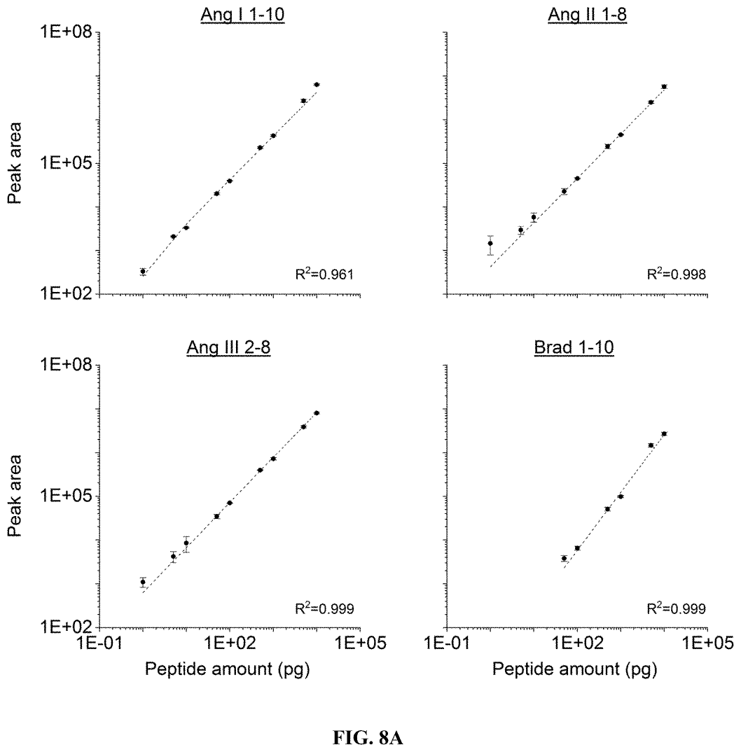

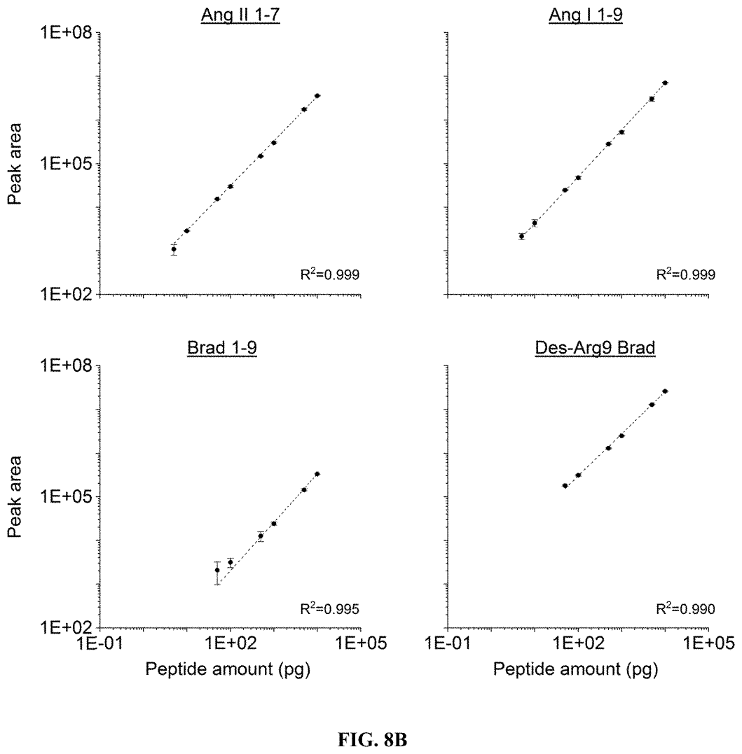

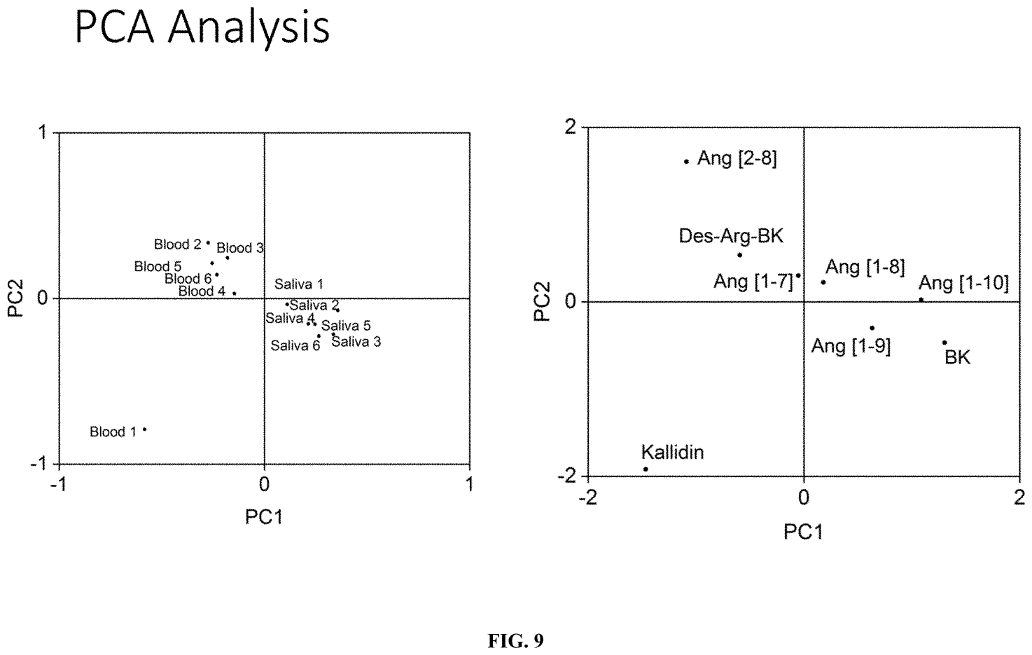

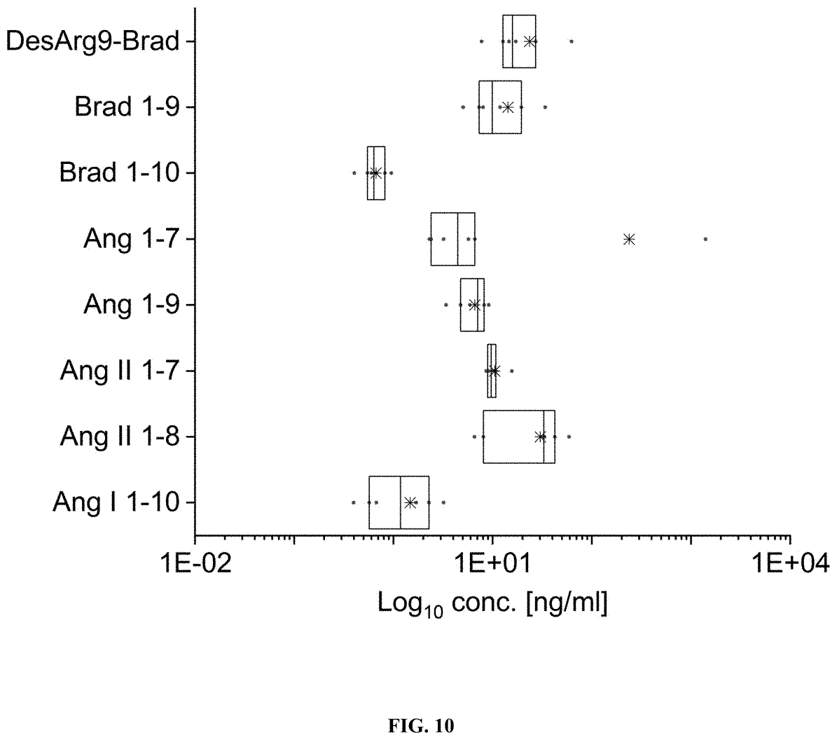

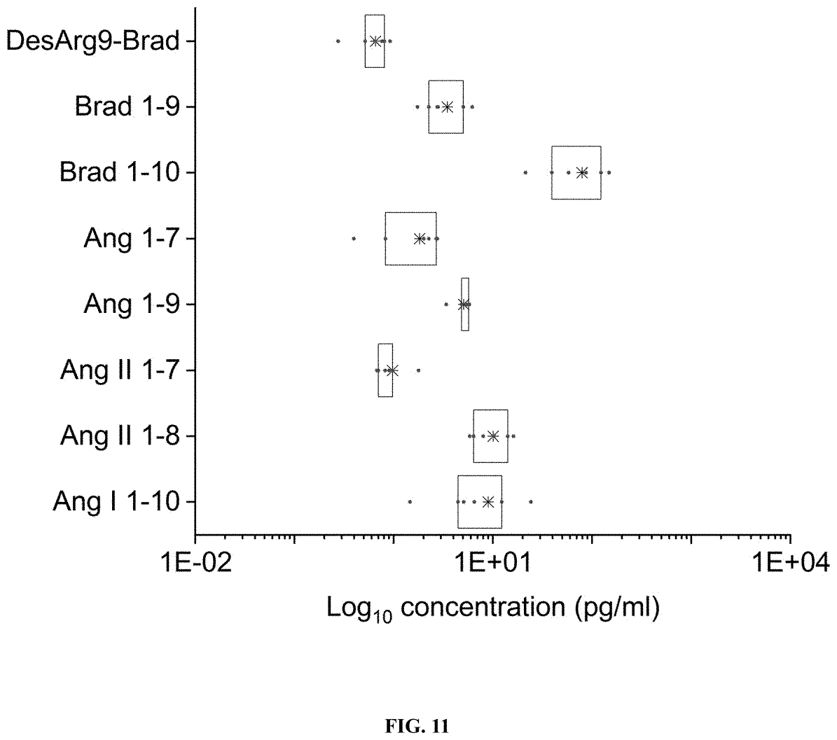

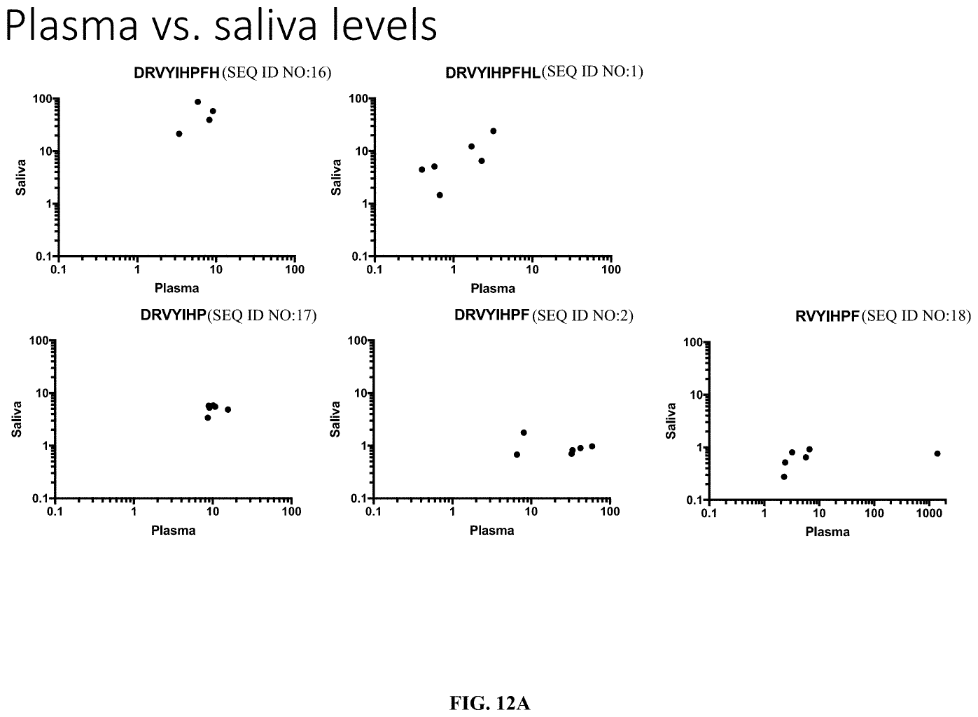

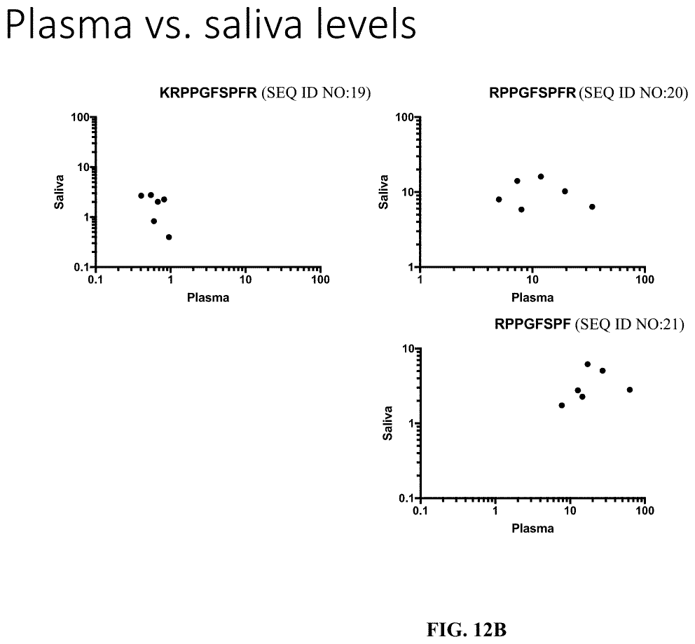

Protein sequence coverage analysis of our Angt and Kng1 proteomics data (FIG. 5) revealed that the respective regions of the precursor proteins that yield the angiotensin and kinin peptides are missing, which indirectly indicates that these are sites of proteolytic processing—which is consistent with the hypothesis of angiotensin and kinin peptide generation in vivo.

TABLE 1
Normalized average RNA transcription in adult and fetal salivary
glands (SM, submandibular; PAR, parotid; SL, sublingual)

| | adult | | | fetal | | |
|---|---|---|---|---|---|---|
| Gene | SM | PAR | SL | SM | PAR | SL |
| REN | 1.42 | 1.44 | 5.64 | 2.03 | 0.00 | 7.17 |
| ANGT | 6.24 | 19.02 | 14.04 | 29.51 | 14.21 | 16.28 |
| ACE | 94.19 | 100.35 | 202.45 | 168.53 | 160.76 | 137.99 |
| ACE2 | 22.82 | 12.29 | 40.30 | 20.18 | 14.13 | 53.29 |
| ANGTR1 | 41.84 | 52.27 | 15.55 | 70.20 | 53.11 | 43.15 |
| ANGTR2 | 1.91 | 2.03 | 3.74 | 401.7 | 350.8 | 328.8 |
| MAS1 | 0.00 | 0.28 | 0.00 | 0.17 | 0.80 | 2.47 |
| KNG1 | 4.07 | 4.92 | 2.44 | 5.95 | 7.81 | 21.31 |

### Example 3. Detecting an Overactive Inflammatory Response in SARS-CoV2 Patients

SARS-CoV has been shown to lead to a loss of ACE2 protein abundance, which in the lung, plays a crucial role in the development of acute respiratory distress syndrome (ARDS) caused by SARS-CoV infection by enhanced activation of the pro-inflammatory AT1 receptor by Ang II (Kuba, K. et al., A crucial role of angiotensin converting enzyme 2 (ACE2) in SARS coronavirus-induced lung injury, Nat Med 11, 875-79 (2005)). ACE2 functions as a potent negative regulator of RAS-induced inflammation mainly due to its deactivation of Ang II to Ang [1-7] which reduces AngII/AT1R signaling and activates the Ang [1-7]/Mas axis. Additionally, ACE2 inactivates Des-Arg9-bradykinin thereby limiting inflammation induced by KS. Treatment with recombinant ACE2 protein improves symptoms of acute lung injury in wild-type and ace2 knockout mice (Paul, M., Wagner, J. & Dzau, V. J., Gene expression of the renin-angiotensin system in human tissues, Quantitative analysis by the polymerase chain reaction. Journal of Clinical Investigation 91: 2058-64 (1993)). Currently, it is unclear whether ACE loss is due to a (1) virus-mediated internalization of the membrane-bound protein, (2) downregulation of gene expression or (3) shedding by proteases from the cell surface. Neither ACE, ACE2 or the receptors have been detected in saliva collected from healthy human donors or in the diseases currently studied. It is unknown whether ACE2 is shed in SARS-CoV infected individuals.

Altogether, ACE2 is a key regulator of the inflammatory response by limiting the overactivation of both RAS and KS and preventing tissue damage caused by an overactive inflammatory response.

A unique saliva and mass spectrometry-based bioassay described herein is used to monitor RAS- and ACE2-related bioactivity in saliva. The bioassay is used to predict a general inflammatory responsiveness of the human host to coronavirus infection by monitoring ACE2 activity and the associated RAS and KS signaling peptides in saliva of infected patients as a key step to aid in the medical treatment decision process.

The immuno-Matrix Assisted Laser Desorption/Ionization (iMALDI) method has been developed using anti-IgG beads to capture anti-AngI and anti-AngII antibodies, which are incubated with a ˜50 μL plasma sample to which known amounts of stable-isotope-labeled AngI and AngII have been added.

The iMALDI assay can detect and quantify angiotensin I (AngI) and angiotensin II (AngII) in human plasma. This assay has a Limit of Detection (LOD) of ˜10 amol/IL (or ˜13 pg/mL AngI and ˜11 pg/mL AngII), at a S/N of 2:1, using only one-tenth of the antibody beads which were incubated with a 50-IL plasma sample. This LOD is within the relevant range of patient samples. Little or no angiotensin generation period is required, resulting in a rapid assay.

Poly-clonal rabbit anti-human angiotensin 1, angiotensin 2, and bradykinin (BK) antibodies (Invitrogen) are conjugated to magnetic protein G dynabeads (Invitrogen). Anti-Ang I, -Ang II, -BK affinity beads are added to saliva samples to which known amounts of stable isotope labeled analogs of the target peptides (Ang I, II, [1-7] etc.) have been added. After a short incubation time, beads are washed and a 1 μL aliquot placed directly on a 384-well MALDI-stainless steel target plate. Once dry, each sample will be coated with 1 μL of alpha-cyano-4-hydoxycinnamic acid (CHCA) in 0.1% trifluoroacetic acid. Samples will be analyzed in a high resolution MALDI TOF/TOF instrument (such as Sciex 4800) by MS and MS/MS data acquisition. MS peak areas will be used for quantitation. Absolute quantities will be determined using a standard curve generated by varying the amount of stable-isotope peptide analogs. This method has been previously described in more detail for quantitation of Ang I and II in plasma (Mason et al. 2012) and is covered by U.S. Pat. No. 7,846,748 B2 and Canadian Patent 2,507,864.

Detection of ACE2-related bioactivity can be alternatively detected by a peptide-cleavage assay in case the enzyme is shed from the epithelial cells. Expected low abundances of salivary angiotensin-peptides may require a prior immunoaffinity step using a pan-angiotensin polyclonal antibody to enrich for the peptides and simplify the analyte prior to detection by LC-MS/MS. In case monoclonal antibodies are capable to differentiate between peptides Ang I [1-10], Ang II, Ang [1-9] and Ang [1-7] (they differ in length by a single or two amino acids) quantitation could be performed using an ELISA-type assay.

### Example 4. Quantitation of Angiotensin and Kinin Peptides in Biofluids

Peptide Extraction.

One ml of sample (plasma, saliva) was mixed with 4 ml methanol and 100 μl internal standards (13C 15N Ang I; 13C 15N AngII; 100 pg/μ1) to facilitate quantification and calculation of recovery. Samples were stored in the freezer at −20° C. for at least 1 h to allow the precipitation of proteins and were then centrifuged. Supernatants were collected and evaporated to complete dryness under nitrogen flow in an automated evaporation system (TurboVap LV, Biotage). Peptides were extracted using 200 mg solid-phase C18 columns (Biotage, Uppsala, Sweden), brought to dryness using the TurboVap and immediately resuspended in 100 ul sample buffer (5% Acetic acid, 2% ACN in ddH₂O) supplemented with 1 fmol/μl PepCalMix quality control standard (SCIEX, Framingham, Mass., USA).

Quantitation by multiple reaction monitoring (MRM) liquid chromatography-tandem mass spectrometry (LC-MS/MS)

A targeted approach using MRM-LC-MS/MS was performed to quantify levels of angiotensin and kinin peptides in plasma and saliva samples. 20 ul of extracted peptides were injected into an LC-MS/MS system comprised of a Shimadzu Nexera XR liquid chromatography system (Shimadzu, Kyoto, Japan) hyphenated to a SCIEX QTRAP 6500 mass spectrometer (SCIEX, Framingham, Mass., USA) equipped with an Ion Drive Turbo V ESI source. Analytes were separated by a linear gradient (Solution A: 0.1% formic acid in ddH₂O; B: 0.1% formic acid in ACN) from 3% up to 30% solvent B over 3.5 min over a Kinetex XB-C18 column (3.0×50 mm, 2.6-micron particle size) column (Phenomenex, Torrance, Calif., USA) guarded by a Security Guard Ultra Peptide C18 precolumn (Phenomenex). The column was subsequently washed for 0.5 min at 90% B and re-equilibrated with 3% solvent A over 1.0 min. The flow rate was 0.5 ml/min and the column was heated to 50° C. To monitor and quantify the peptide levels, an MRM was developed with signature ion fragments for each molecule (See Table 1). Calibration curves for each analyte were obtained at 0.1, 1, 10, 100, and 1,000 pg/μ1 using injection volumes of 5 and 10 μl with r² values in the range 0.98-0.99. Quantification was carried out based on extracted chromatographic peak areas of the MRM transitions and the linear calibration curve for each compound using internal standards for correction of sample losses and matrix effects during sample preparation and acquisition of mass spectrometry data.

TABLE 2
Substance standards for the quantitation of RAS/kinin peptides.

| | Standard Concentration | | | |
|---|---|---|---|---|
| | | internal | internal | external |
| | diluted | quantitation | QC | QC |
| Compound | [pg/μl] | [pg/μl] | [fmol/μl] | [pg/μl] |
| Angiotensin I | 0.1-1000 | — | — | 10 |
| Angiotensin II | 0.1-1000 | — | — | 10 |
| Angiotensin [1-7] | 0.1-1000 | — | — | 10 |
| Angiotensin [1-9] | 0.1-1000 | — | — | 10 |
| Angiotensin III | 0.1-1000 | — | — | 10 |
| Bradykinin | 0.1-1000 | — | — | 10 |
| Kallidin | 0.1-1000 | — | — | 10 |
| (Des-Arg9)-Bradykinin | 0.1-1000 | — | — | 10 |
| 13C 15N Angiotensin I | 0.1-1000 | 100 | — | 10 |
| 13C 15N Angiotensin II | 0.1-1000 | 100 | — | 10 |
| PepCalMix.GAYVEVTAK. + 2y5.heavy | — | — | 1 | — |
| PepCalMix.SAEGLDASASLR. + 2y7.heavy | — | — | 1 | — |
| PepCalMix.VGNEIQYVALR. + 2y6.heavy | — | — | 1 | — |
| PepCalMix.YIELAPGVDNSK. + 2y7.heavy | — | — | 1 | — |
| PepCalMix.VGNEIQYVALR. + 2y6.heavy | — | — | 1 | — |

TABLE 3
HPLC method for the separation of RAS/kinin peptides.

| Time | Module | Event | Parameter |
|---|---|---|---|
| 0.10 | Pumps | Pump B. Conc | 3 |
| 3.50 | Pumps | Pump B. Conc | 30 |
| 3.51 | Pumps | Pump B. Conc | 90 |
| 4.00 | Pumps | Pump B. Conc | 90 |
| 4.01 | Pumps | Pump B. Conc | 3 |
| 5.00 | Pumps | Pump B. Conc | 3 |
| 5.01 | Controller | Stop | |

### Example 5. Dysregulated Angiotensin and Kinin Profiles in COVID-19

Clinical presentation of coronavirus disease 2019 (COVID-19), the illness caused by the novel severe acute respiratory syndrome coronavirus 2 (SARS-CoV-2), can range from asymptomatic to mild infections to severe disease that can progress to acute respiratory distress syndrome (ARDS) and multiorgan failure leading to death (Guan et al., N Engl J Med. 382(18):1708-20 (2020)). Currently, there are no specific treatments for COVID-19 and the molecular details of how SARS-CoV-2 manipulates the host immune response are not fully characterized. Both SARS-CoV-1 and -2 use angiotensin-converting enzyme 2 (ACE2) for cell entry (Hoffmann et al., Cell 181(2):271-80 (2020)). SARS-CoV-2 infection is hypothesized to directly impact inflammatory pathways by perturbating the activity of the ACE2 host peptidase by depleting ACE2 from the host cell plasma membrane by either increased endocytosis or increased shedding (Kuba et al., Nat Med. 11(8):875-9 (2005), Lambert et al., J Biol Chem. 280(34):30113-9 (2005), Heurich et al., J Virol. 88(2):1293-307 (2014), and Meini et al., Front Immunol. 11:2014 (2020)). ACE2 is a critical peptidase of two host regulatory pathways, the renin-angiotensin system (RAS) and the kinin-kallikrein system (KKS). In the RAS, ACE2 controls the abundance of angiotensin II (Ang[1-8]) the principal effector of RAS that signals through the pro-inflammatory AT1 receptor and generates the organ-protective Ang[1-7] that signals via the Mas receptor (FIG. 3A). In the KKS, ACE2 controls the abundance of the pro-inflammatory des-Arg(9)-bradykinin (BK[1-8]) that signals via B1R (FIG. 3B). SARS-CoV-mediated loss of ACE2 function has been hypothesized to impair the deactivation of Ang[1-8] and BK[1-8] and decreased levels of the protective Ang[1-7]. Accumulation of bradykinin (BK[1-9]) and BK[1-8] are predicted to trigger a positive feedback loop of inflammation, coughing and injury that could contribute to pulmonary angioedema (Roche & Roche R. FASEB J. 34(6):7265-69 (2020), van de Veerdonk et al., Elife. 9:e57555 (2020), de Maat et al., Semin Thromb Hemost. 46(7):835-37 (2020)). Exploratory treatment with B2-receptor antagonist icatibant improved oxygenation in a case-control study (van de Veerdonk et al., JAMA Netw Open. 3(8):e2017708 (2020)).

Clinical manifestations of COVID-19 are characterized by coagulopathy evidenced by microthrombosis and high levels of D-dimer which indicates hyperactivation of both coagulation and fibrinolysis. Therefore, the contact system in the vasculature is emerging as a key factor in the innate immune response linking the activation of the KKS with coagulation, fibrin formation and fibrinolysis as well as a negative feedback via the RASE.

Measurements of RAS and KKS peptide abundances are not standard clinical procedure. Many of the RAS/KKS peptides differ only by a single added amino acid which renders their detection by immunoassays challenging due to cross-reactivity. While highly specific and sensitive liquid-chromatography-tandem mass spectrometry (LC-MS/MS)-based assays have been developed to detect RAS peptides in plasma/serum and most recently for KKS peptides in nasal lavage fluid (Gangnus & Burckhardt, Sci Rep. 11(1):3061 (2020)), comprehensive measurements in COVID-19 patients are currently lacking and the role of RAS and KKS in COVID-19 has not been elucidated.

Materials and Methods

Ethics Statement. The study was approved by the Kuwait Ministry of Health and the Forsyth Institute Institutional Review Board. Informed consents were obtained from all enrolled participants.

Study design and population. Patients were recruited among those admitted at multiple hospital sites (AlFarwania Hospital, Jaber Al Ahmed Hospital, Kuwait Field Hospital) in the State of Kuwait between Jul. 24 and Sep. 4, 2020 that tested positive for SARS-CoV-2 by RT-PCR from nasopharyngeal swabs (n=30). Patients in this study were enrolled as part of a Kuwait Ministry of Health-COVID19 biorepository study after obtaining informed consent. Non-infected control samples (plasma, saliva) were obtained from study participants who were not diagnosed with SARS-CoV-2 (n=10). Basic demographics and clinical information of study participants were obtained and shown in Table 4. Severity of COVID-19 was assessed according to the following categories: control: uninfected, no evidence of infection; mild: hospitalized, but no oxygen therapy; moderate: hospitalized, low-flow oxygen (≤10 L/min); severe: hospitalized, high flow oxygen (>10 L/min).

TABLE 4
MRM transitions for the detection of the targeted
RAS and KKS peptides and quality control standards

| | | RT | | Q1 | Q3 | DP | CE |
|---|---|---|---|---|---|---|---|
| Peptide | Abbrev. | [min] | Ion | [m/z] | [m/z] | [V] | [V] |
| Angiotensin I | Ang[1-10] | 3.4 | MH3+ | 432.9 | 109.9 | 80 | 22 |
| Angiotensin I | Ang[1-10] | 3.4 | MH3+ | 432.9 | 513.5 | 80 | 22 |
| Angiotensin I | Ang[1-10] | 3.4 | MH3+ | 432.9 | 647.4 | 80 | 22 |
| Angiotensin I | Ang[1-10] | 3.4 | MH3+ | 432.9 | 393.0 | 80 | 22 |
| Angiotensin I | Ang[1-10] | 3.4 | MH3+ | 432.9 | 86.1 | 80 | 22 |
| Angiotensin II | Ang[1-8] | 3.2 | MH2+ | 523.8 | 263.1 | 80 | 30 |
| Angiotensin II | Ang[1-8] | 3.2 | MH2+ | 523.8 | 784.4 | 80 | 30 |
| Angiotensin II | Ang[1-8] | 3.2 | MH2+ | 523.8 | 756.4 | 80 | 30 |
| Angiotensin II | Ang[1-8] | 3.2 | MH2+ | 523.8 | 109.9 | 80 | 30 |
| Angiotensin II | Ang[1-8] | 3.2 | MH2+ | 523.8 | 392.8 | 80 | 30 |
| Angiotensin [1-7] | Ang[1-7] | 2.5 | MH2+ | 450.2 | 647.3 | 80 | 28 |
| Angiotensin [1-7] | Ang[1-7] | 2.5 | MH2+ | 450.2 | 619.4 | 80 | 28 |
| Angiotensin [1-7] | Ang[1-7] | 2.5 | MH2+ | 450.2 | 534.3 | 80 | 28 |
| Angiotensin [1-7] | Ang[1-7] | 2.5 | MH2+ | 450.2 | 110.0 | 80 | 28 |
| Angiotensin [1-7] | Ang[1-7] | 2.5 | MH2+ | 450.2 | 116.1 | 80 | 28 |
| Angiotensin [1-9] | Ang[1-9] | 2.8 | MH3+ | 395.2 | 110.1 | 80 | 30 |
| Angiotensin [1-9] | Ang[1-9] | 2.8 | MH3+ | 395.2 | 156.1 | 80 | 30 |
| Angiotensin [1-9] | Ang[1-9] | 2.8 | MH3+ | 395.2 | 619.5 | 80 | 30 |
| Angiotensin [1-9] | Ang[1-9] | 2.8 | MH3+ | 395.2 | 534.1 | 80 | 30 |
| Angiotensin [1-9] | Ang[1-9] | 2.8 | MH3+ | 395.2 | 303.3 | 80 | 30 |
| Angiotensin III | Ang[2-8] | 3.0 | MH2+ | 466.3 | 263.1 | 80 | 25 |
| Angiotensin III | Ang[2-8] | 3.0 | MH2+ | 466.3 | 669.4 | 80 | 25 |
| Angiotensin III | Ang[2-8] | 3.0 | MH2+ | 466.3 | 641.4 | 80 | 25 |
| Angiotensin III | Ang[2-8] | 3.0 | MH2+ | 466.3 | 523.3 | 80 | 25 |
| Angiotensin III | Ang[2-8] | 3.0 | MH2+ | 466.3 | 110.0 | 80 | 25 |
| Bradykinin | BK[1-9] | 2.9 | MH2+ | 530.8 | 904.5 | 80 | 30 |
| Bradykinin | BK[1-9] | 2.9 | MH2+ | 530.8 | 156.9 | 80 | 30 |
| Bradykinin | BK[1-9] | 2.9 | MH2+ | 530.8 | 174.9 | 80 | 30 |
| Bradykinin | BK[1-9] | 2.9 | MH2+ | 530.8 | 452.8 | 80 | 30 |
| Bradykinin | BK[1-9] | 2.9 | MH2+ | 530.8 | 807.5 | 80 | 30 |
| Kallidin | K-BK[1-9] | 2.6 | MH2+ | 396.9 | 391.4 | 80 | 20 |
| Kallidin | K-BK[1-9] | 2.6 | MH2+ | 396.9 | 419.3 | 80 | 25 |
| Kallidin | K-BK[1-9] | 2.6 | MH2+ | 396.9 | 506.3 | 80 | 25 |
| Kallidin | K-BK[1-9] | 2.6 | MH2+ | 396.9 | 210.2 | 80 | 25 |
| Kallidin | K-BK[1-9] | 2.6 | MH2+ | 396.9 | 120.0 | 80 | 25 |
| (Des-Arg9)-Bradykinin | BK[1-8] | 3.4 | MH2+ | 452.7 | 263.2 | 80 | 25 |
| (Des-Arg9)-Bradykinin | BK[1-8] | 3.4 | MH2+ | 452.7 | 624.4 | 80 | 25 |
| (Des-Arg9)-Bradykinin | BK[1-8] | 3.4 | MH2+ | 452.7 | 70.0 | 80 | 25 |
| (Des-Arg9)-Bradykinin | BK[1-8] | 3.4 | MH2+ | 452.7 | 614.4 | 80 | 25 |
| (Des-Arg9)-Bradykinin | BK[1-8] | 3.4 | MH2+ | 452.7 | 552.2 | 80 | 25 |
| Bradykinin [1-7] | BK[1-7] | 2.5 | MH2+ | 379.2 | 203.1 | 80 | 20 |
| Bradykinin [1-7] | BK[1-7] | 2.5 | MH2+ | 379.2 | 321.7 | 80 | 20 |
| Bradykinin [1-7] | BK[1-7] | 2.5 | MH2+ | 379.2 | 370.2 | 80 | 20 |
| Bradykinin [1-7] | BK[1-7] | 2.5 | MH2+ | 379.2 | 70 | 80 | 20 |
| Bradykinin [1-7] | BK[1-7] | 2.5 | MH2+ | 379.2 | 116 | 80 | 20 |
| Bradykinin [1-5] | BK[1-5] | 2.4 | MH2+ | 287.2 | 254.1 | 80 | 20 |
| Bradykinin [1-5] | BK[1-5] | 2.4 | MH2+ | 287.2 | 166 | 80 | 20 |
| Bradykinin [1-5] | BK[1-5] | 2.4 | MH2+ | 287.2 | 120 | 80 | 20 |
| Bradykinin [1-5] | BK[1-5] | 2.4 | MH2+ | 287.2 | 70 | 80 | 20 |
| Bradykinin [1-5] | BK[1-5] | 2.4 | MH2+ | 287.2 | 162.1 | 80 | 20 |
| ¹³C, ¹⁵N-Angiotensin I | Ang[1-10]* | 3.4 | MH3+ | 437.2 | 109.9 | 80 | 22 |
| ¹³C, ¹⁵N-Angiotensin I | Ang[1-10]* | 3.4 | MH3+ | 437.2 | 513.5 | 80 | 22 |
| ¹³C, ¹⁵N-Angiotensin I | Ang[1-10]* | 3.4 | MH3+ | 437.2 | 660.4 | 80 | 22 |
| ¹³C, ¹⁵N-Angiotensin I | Ang[1-10]* | 3.4 | MH3+ | 437.2 | 399.1 | 80 | 22 |
| ¹³C, ¹⁵N-Angiotensin I | Ang[1-10]* | 3.4 | MH3+ | 437.2 | 86.1 | 80 | 22 |
| ¹³C, ¹⁵N-Angiotensin II | Ang[1-8]* | 3.2 | MH2+ | 527.3 | 263.1 | 80 | 30 |
| ¹³C, ¹⁵N-Angiotensin II | Ang[1-8]* | 3.2 | MH2+ | 527.3 | 791.5 | 80 | 30 |
| ¹³C, ¹⁵N-Angiotensin II | Ang[1-8]* | 3.2 | MH2+ | 527.3 | 763.3 | 80 | 30 |
| ¹³C, ¹⁵N-Angiotensin II | Ang[1-8]* | 3.2 | MH2+ | 527.3 | 109.9 | 80 | 30 |
| ¹³C, ¹⁵N-Angiotensin II | Ang[1-8]* | 3.2 | MH2+ | 527.3 | 396.1 | 80 | 30 |
| PepCalMix.GAYVEVTAK. + 2y5.heavy | | 2.5 | MH2+ | 473.3 | 555.3 | 80 | 20 |
| PepCalMix.SAEGLDASASLR. + 2y7.heavy | | 2.8 | MH2+ | 593.8 | 729.4 | 80 | 29 |
| PepCalMix.VGNEIQYVALR. + 2y6.heavy | | 3.3 | MH2+ | 636.4 | 759.4 | 80 | 31 |
| PepCalMix.YIELAPGVDNSK. + 2y7.heavy | | 3.2 | MH2+ | 657.3 | 724.4 | 80 | 27 |
| PepCalMix.SPYVITGPGWEYK. + 2y9.heavy | | 3.5 | MH2+ | 758.9 | 957.5 | 80 | 38 |

RT, retention time; DP, declustering potential; CE, collision energy; [m/z] mass-over-charge. The RAS-KKS peptide QC standard contained synthetic analogs of the analytes at 0.25 pg/μl except for BK[1-8] at 0.05 pg/μl and both BK[1-9] and K-BK[1-9] at 5 pg/μl.

Sample Collection. Saliva and plasma were collected at the time of hospital admission (viral testing) and on a weekly schedule for the subsequent 14 days, except for uninfected controls for who no third sample was obtained. Sample collection and isolation were performed according to standard protocols. Stimulated whole saliva (3-4 mL) was collected over a 10-min period and kept on ice. Patient blood was collected in BD Vacutainer (EDTA) tubes (Becton Dickenson, Franklin Lakes, N.J., USA). Plasma fractions were collected after centrifugation at 2000×g at room temperature for 10 min. Both plasma and saliva samples were aliquoted (500 μL) and viral inactivated by the addition of cold methanol (final concentration: 75%), mixed and stored at −80° C. until use.

Chemicals and Reagents. LC-MS-grade acetonitrile, methanol, isopropanol and water were purchased from Fisher Scientific (Hampton, N.H., USA). Formic acid (>99% purity) was supplied by Thermo Scientific (Waltham, Mass., USA). Peptide standards (Angiotensin I (Ang[1-10]), angiotensin II (Ang[1-8], Ang[1-9], angiotensin III (Ang[2-8], bradykinin (BK[1-9], kallidin (K-BK[1-9]), (Des-Arg9)-Bradykinin (BK[1-8]), BK[1-7] and BK[1-5] and ¹³C, ¹⁵N-labeled Ang[1-10]*) and ¹³C, ¹⁵N-labeled Ang[1-8]* internal standards were obtained from Bachem (Torrance, Calif., USA) and AnaSpec (Fremont, Calif., USA).

Peptide extraction. Methanolic plasma and saliva samples (2 mL) were spiked with 2.5 ng of stable isotope-labeled internal standards (¹³C, ¹⁵N-labeled Ang[1-10] and ¹³C, ¹⁵N-labeled Ang[1-8]). Samples were stored at −20° C. for 1 hour to allow for precipitation of proteins prior to centrifugation at 20,000×g for 10 min at 4° C. Supernatants were evaporated to complete dryness under nitrogen flow in an automated evaporation system (TurboVap LV, Biotage, Uppsala, Sweden). Samples were resuspended in 1 ml 0.5% TFA, centrifuged for 10 min at 2500×g, and the supernatants were subjected to solid phase extraction using filter columns with 200 mg C18 resin (Biotage, Uppsala, Sweden), washed twice with 3 ml 0.1% formic acid in water, eluted with 1 ml 0.1% formic acid in 50% ACN, evaporated, and resuspended in 50 sample buffer (0.1% formic acid in ddH₂O) supplemented with 1 fmol/μ1 PepCalMix quality control (QC) standard (SCIEX, Framingham, Mass., USA). To remove insoluble material, the centrifugation step was repeated prior to LC-MS/MS analysis.

Targeted LC-MS/MS-analysis by multiple-reaction monitoring (MRM). Targeted quantitation experiments were performed at the Forsyth Center for Salivary Diagnostics on a SCIEX QTRAP 6500 triple quadrupole mass spectrometer (SCIEX, Framingham, Mass., USA) equipped with an Ion Drive Turbo V ESI source hyphenated to a Shimadzu Nexera XR HPLC system (Shimadzu, Kyoto, Japan). A Kinetex XB-C18 column (3.0×50 mm, 2.6-micron particle size; Phenomenex, Torrance, Calif., USA) guarded by a Security Guard Ultra Peptide C18 precolumn (Phenomenex) was used for HPLC separation. The mass spectrometer was optimized for the detection of each individual peptide to obtain maximum intensity (polarity, collision energy, precursor and product ion selection) and the parameters are summarized in Supplemental Table 1. The injection volume of peptide extracts was 20 μl. Peptide analytes were separated using a linear gradient (Solution A: 0.1 formic acid in ddH₂O; B: 0.1% formic acid in ACN) from 3% up to 30% solvent B over 3.5 min). The column was subsequently washed for 0.5 min at 90% B and re-equilibrated with 3% solvent A over 1.0 min. The flow rate was 0.5 mL/min and the column was heated to 50° C. The ESI source nebulizer and heater nitrogen gas was adjusted to a flow of 65 (arbitrary units), while the curtain gas was maintained at 30 (arbitrary units). The ion source temperature was maintained at 500° C. with a voltage of 5500 V in positive ionization mode. Samples were analyzed in randomized order. The PepCalMix quality control (QC) standard as well as a RAS-KKS peptide QC standard containing synthetic analogs of all analytes were analyzed at predefined intervals during and between analysis batches to monitor the stability of the analytical system and detect potential batch effects.

Preparation of calibration standards for quantification. Calibration curves for each analyte were obtained at 0.01, 0.1, 1, 10, 100, and 1,000 μg per injection using volumes of 54, and 10 μL with r² values in the range 0.98-0.99. All stock solutions were aliquoted and stored at −20° C. until used. MultiQuant 3.0.1 software (SCIEX) was used to extract peak areas from the chromatographic data. Quantification was achieved for each analyte by using linear regression analysis of peak areas versus the concentration of the analytes.

Data processing and statistical analysis. MRM-data was processed using MultiQuant 3.0.1 software (SCIEX) to detect and quantify the targeted peptide using the respective transition settings listed for each peptide analyte in Table 4. Quantification was carried out based on extracted chromatographic peak areas of the MRM transitions and the linear calibration curve for each peptide. Internal standards (¹³C, ¹⁵N-Ang[1-10] and ¹³C, ¹⁵N-Ang[1-8]) at a concentration of 50 pg/μl were used to correct for analyte recovery across the sample preparation process and matrix effects for individual samples. A custom R script entailing Wilcoxon tests were used for statistical comparisons and resulting p-Values were adjusted using the Benjamini and Hochberg method (Benjamini & Hochberg, Journal of the Royal Statistical Society. Series B (Methodological) 57(1):289-300 (1995)).

Results and Discussion

A targeted LC-MS/MS workflow was developed for the detection of RAS and KKS peptides to determine whether interference of SARS-CoV-2 with ACE2 function leads to the predicted alterations of the host RAS/KKS pathways and whether RAS/KKS peptide levels correlate with COVID-19 disease severity. To gain mechanistic understandings on how SARS-CoV-2 dysregulates RAS/KKS signaling at the site of initial infection (oral cavity) and systemically, serial saliva and serum samples were collected from 10 healthy individuals with negative viral RT-PCR test (control), 10 COVID-19 patients (positive viral RT-PCR test) with mild symptoms and no need for oxygen supplementation, 10 COVID-19 patients with moderate symptoms requiring low flow oxygen supplementation and 10 COVID-19 patients with severe symptoms requiring high flow oxygen supplementation. The demographics of these patients are shown in Table 5. For biosafety, samples were viral inactivated by the addition of 3× methanol which simultaneously served as deproteinization step of the peptide extraction method prior to the measurement of absolute peptide quantities by LC-MS/MS.

TABLE 5
Patient Characteristics

| | Control | Mild | Moderate | Severe |
|---|---|---|---|---|
| | N = 10 | N = 10 | N = 10 | N = 10 |
| O₂-supplementation (l/min), mean ± SD | | | 4.7 ± 3.0 | 21.7 ± 10.1 |
| Age, mean ± SD | 36.2 ± 8.5 | 42.7 ± 11.8 | 48.0 ± 14.3 | 54.4 ± 9.0 |
| Male sex (percent) | 50% | 50% | 70% | 70% |
| Waist circumference (cm), mean ± SD | 103.7 ± 29.1 | 102.4 ± 16.7 | 112.3 ± 15.6 | 111.4 ± 11.0 |

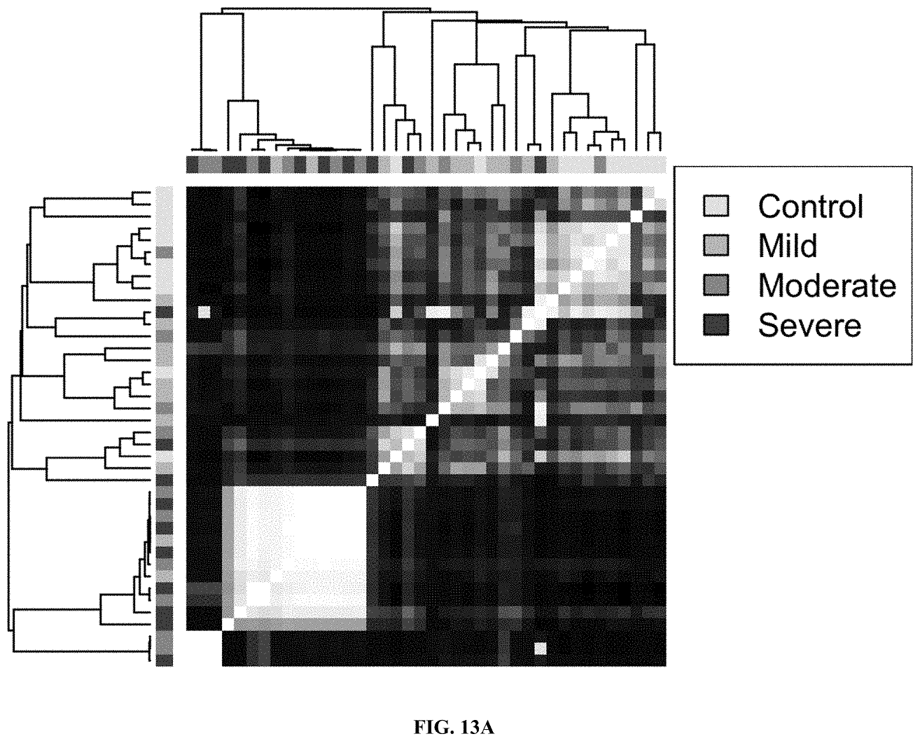

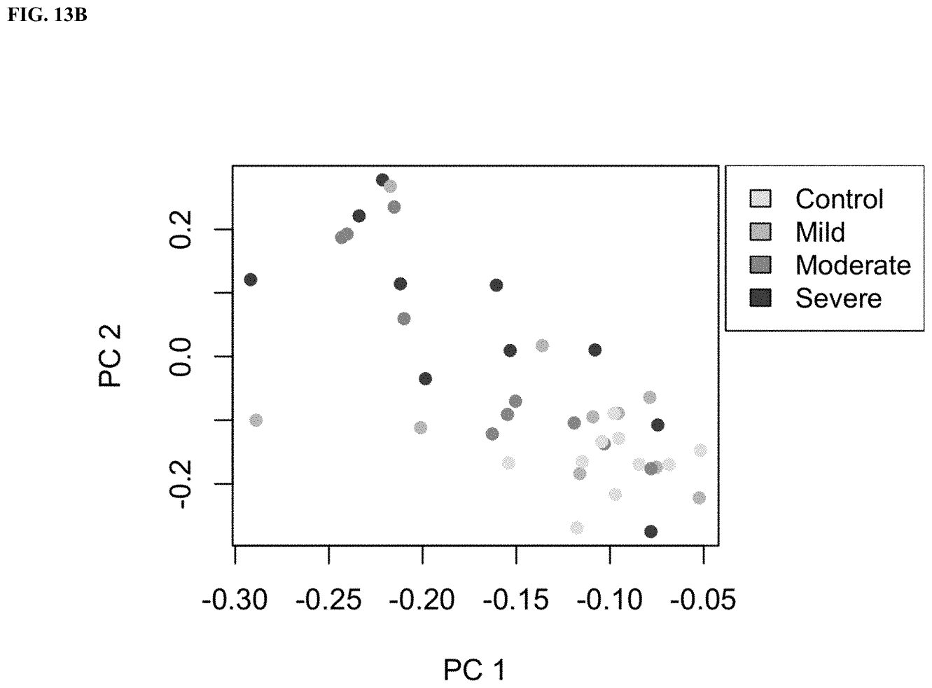

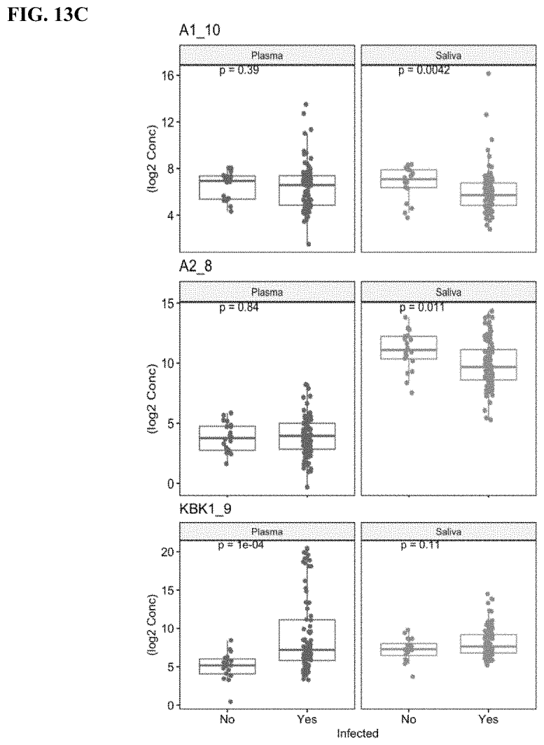

To test the hypothesis that SARS-CoV-2 infection leads to a dysregulation of the RAS and KKS pathways, peptide profiles obtained from uninfected controls and infected patients independent of disease severity or sampling day were compared. In saliva, SARS-CoV-2-infection was associated with a statistically significant (adj. p=0.0083) reduction of Angiotensin I (Ang[1-10]) and Angiotensin III (Ang[2-8]; adj. p=0.022) levels compared to controls, while in plasma no such change occurred (FIG. 13C). In plasma, SARS-CoV-2-infection was associated with a highly statistically significant (adj. p=0.0002) increase in Kallidin levels (K-BK[1-9]), while there was no significant increase in saliva. Taking day of visit into consideration, lower levels of Ang[1-10] in saliva were only significant (adj. p=0.027) at time of the initial visit. Plasma Kallidin levels (K-BK[1-9]) were significantly different between infected and non-infected patients for both initial visits (adj. p=0.0051 visit 1; adj. p=0.0011 visit 2). No other peptide (including Ang[2-8]) was significantly altered in abundance for any individual visit according to infection status. The findings suggest a temporal association with SARS-CoV-2 infection causing an early dysregulation of RAS in the oral cavity and KKS systemically. Importantly, both Ang[1-10] and K-BK[1-9] appear to be restoring to levels comparable to uninfected controls by the third visit.

Next, how differences in RAS/KKS profiles may be associated with disease severity was explored. Hierarchical clustering (FIG. 13A) revealed that patients who received supplemental oxygen (moderate and severe cases) clustered within the same hierarchical groups and so did for the greatest extend uninfected controls. Principal component analysis of the RAS/KKS profiles showed separation of the severe and moderate COVID-19-cases from the uninfected controls (FIG. 13B).

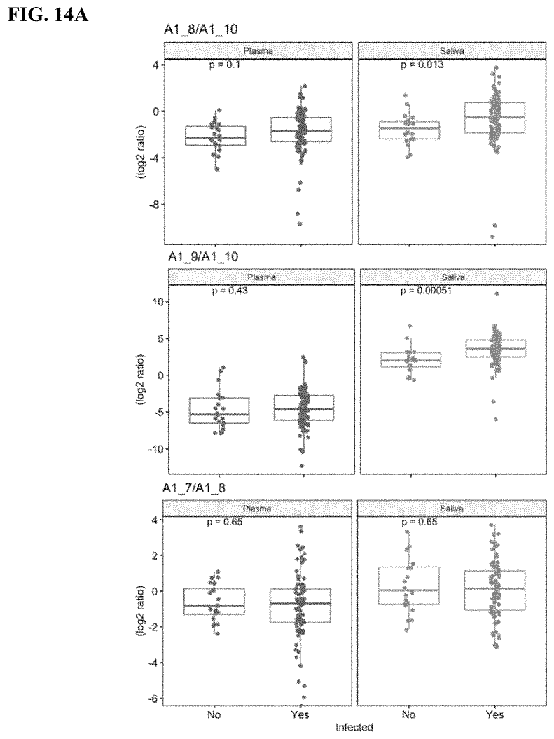

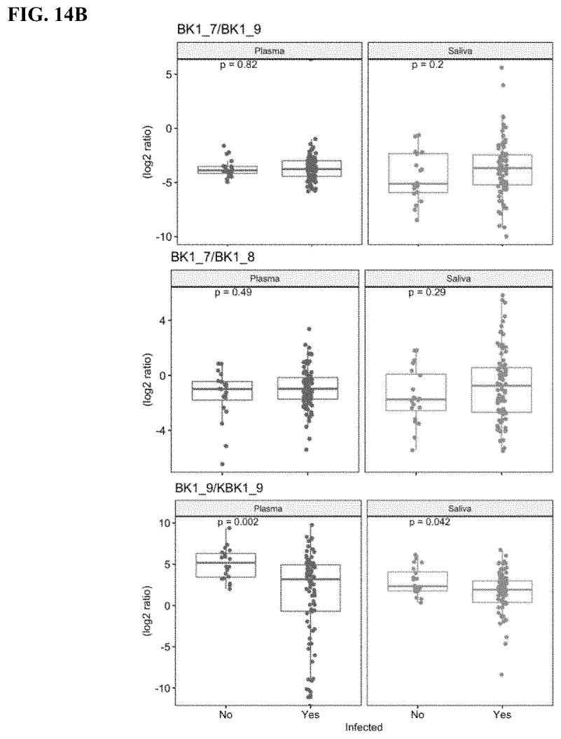

Changes in peptide product levels reflect changes in proteolytic enzyme activity and substrate levels or both. Individual peptide ratios (product/substrate) can serve as activity surrogate measurement for particular peptides. By comparing the abundances of the respective enzymatic substrates and products (e.g., Ang[1-10] and Ang[1-8]) a surrogate measure for the activity of the responsible peptidase (e.g., ACE) can be deduced. In saliva, ACE and ACE2 surrogate activities (Ang[1-8]/Ang[1-10]) (Ang[1-9]/Ang[1-10]), respectively, were significantly upregulated (adj. p=0.025 and adj. p=0.001) in infected individuals (FIG. 14A). This observation is likely due to the aforementioned decreased levels of Ang[1-10] rather than changes in enzymatic activity as other ACE and ACE2 surrogates in saliva failed to show significant alterations. Elevated plasma levels of Ang[1-10] have been associated with non-survival in acute respiratory distress syndrome (Reddy et al., PLoS One. 14(3):e0213096 (2019)). Such an increase was not detected in plasma collected from SARS-CoV-2-infected patients suggesting a distinct mechanism is at play in COVID-19. The observed decreased levels of salivary Ang[1-10] in our study suggest a reduction of renin or renin-like activity in the oral cavity or decreased salivary secretion of the precursor protein angiotensinogen in infected individuals. Ang[1-10] does not have any known biological functions and its generation is in general considered the rate-limiting step of the RAS.

In the KKS, significant changes to ACE and ACE2 surrogate activities (BK[1_7]/BK[1-9] and BK[1_8]/BK[1-9], respectively) were not observed in saliva and plasma collected from infected patients, which is consistent with the results from RAS that neither ACE nor ACE2 activity was altered by infection. Surrogate activity levels for aminopeptidase M, which catalyzes the conversion of kallidin (K-BK[1-9] to bradykinin (BK[1-9]) was, as expected from the overabundance of plasma kallidin, significantly down (adj. p=0.0033). The observation that APM surrogate activity was also down in saliva collected from infected individuals (adj. p=0.042), suggested that reduced overall APM activity may be at least partially responsible for the accumulation of kallidin in COVID-19 patients. Alternatively, increased cleavage of low molecular weight kinin by tissue kallikrein could be responsible for increased kallidin amounts, or impeded processing of kallidin by carboxypeptidase M (FIGS. 14A-14B).

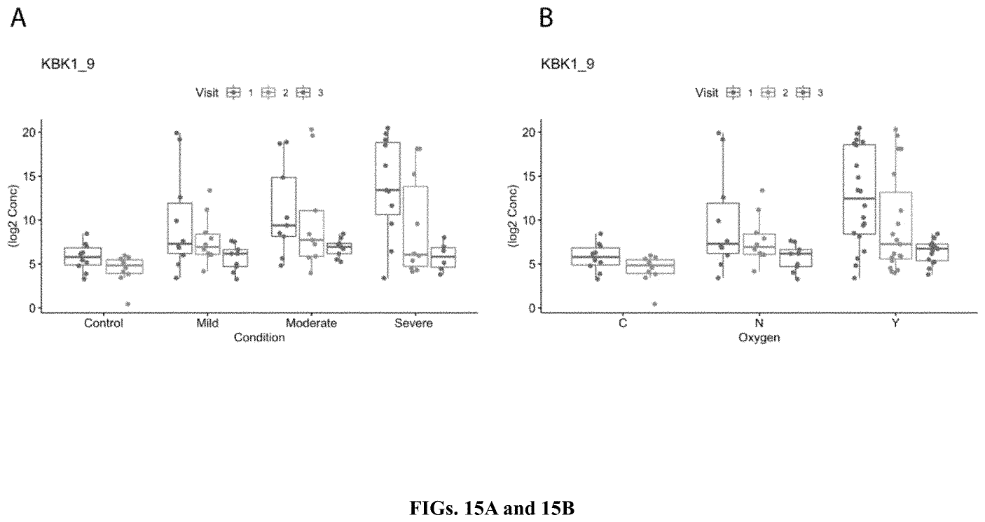

Finally, whether levels of individual RAS/KKS peptides correlated with disease severity and could potentially be used as prognostic markers for disease progression were investigated. It was determined that plasma kallidin levels at visit 1 (time of viral diagnosis) positively correlated with disease severity (FIG. 15A). Kallidin levels were significantly elevated at V1 for moderate (adj. p=0.076) and severe cases (adj. p=0.0076) and for mild (adj. p=0.0019) and moderate (adj. p=0.012) at V2 compared to uninfected controls. Interestingly, across the spectrum of disease severity, kallidin levels, on average, decreased from an initial highpoint at V1 in subsequent visits, suggesting a temporal spike in kallidin levels at the early stages of COVID-19 that correlates with disease severity but tapers off over the course of the disease. Similarly, plasma kallidin levels at V1 are significantly elevated (adj. p=0.0015) in individuals that will receive oxygen supplementation as part of their treatment (FIG. 15B).

### REFERENCES

1. Guan W J, Ni Z Y, Hu Y, Liang W H, Ou C Q, He J X, Liu L, Shan H, Lei C L, Hui DSC, Du B, Li L J, Zeng G, Yuen K Y, Chen R C, Tang C L, Wang T, Chen P Y, Xiang J, Li S Y, Wang J L, Liang Z J, Peng Y X, Wei L, Liu Y, Hu Y H, Peng P, Wang J M, Liu J Y, Chen Z, Li G, Zheng Z J, Qiu S Q, Luo J, Ye C J, Zhu S Y, Zhong N S; China Medical Treatment Expert Group for Covid-19. Clinical Characteristics of Coronavirus Disease 2019 in China. N Engl J Med. 382(18):1708-20 (2020).
2. Hoffmann M, Kleine-Weber H, Schroeder S, Krüger N, Herrler T, Erichsen S, Schiergens T S, Herrler G, Wu N H, Nitsche A, Müller M A, Drosten C, Pöhlmann S. SARS-CoV-2 Cell Entry Depends on ACE2 and TMPRSS2 and Is Blocked by a Clinically Proven Protease Inhibitor. Cell 181(2):271-80 (2020).
3. Kuba K, Imai Y, Rao S, Gao H, Guo F, Guan B, Huan Y, Yang P, Zhang Y, Deng W, Bao L, Zhang B, Liu G, Wang Z, Chappell M, Liu Y, Zheng D, Leibbrandt A, Wada T, Slutsky A S, Liu D, Qin C, Jiang C, Penninger J M. A crucial role of angiotensin converting enzyme 2 (ACE2) in SARS coronavirus-induced lung injury. Nat Med. 11(8):875-9 (2005).
4. Lambert D W, Yarski M, Warner F J, Thornhill P, Parkin E T, Smith A I, Hooper N M, Turner A J. Tumor necrosis factor-alpha convertase (ADAM17) mediates regulated ectodomain shedding of the severe-acute respiratory syndrome-coronavirus (SARS-CoV) receptor, angiotensin-converting enzyme-2 (ACE2). J Biol Chem. 280(34):30113-9 (2005).
5. Heurich A, Hofmann-Winkler H, Gierer S, Liepold T, Jahn O, Pohlmann S. TMPRSS2 and ADAM17 cleave ACE2 differentially and only proteolysis by TMPRSS2 augments entry driven by the severe acute respiratory syndrome coronavirus spike protein. J Virol.
6. Meini S, Zanichelli A, Sbrojavacca R, Iuri F, Roberts A T, Suffritti C, Tascini C.

Understanding the Pathophysiology of COVID-19: Could the Contact System Be the Key? Front Immunol. 11:2014 (2020).
7. Roche J A, Roche R. A hypothesized role for dysregulated bradykinin signaling in COVID-19 respiratory complications. FASEB J. 34(6):7265-69 (2020).
8. van de Veerdonk F L, Netea M G, van Deuren M, van der Meer J W, de Mast Q, Bruggemann R J, van der Hoeven H. Kallikrein-kinin blockade in patients with COVID-19 to prevent acute respiratory distress syndrome. Elife. 9:e57555 (2020).
9. de Maat S, de Mast Q, Danser A H J, van de Veerdonk F L, Maas C. Impaired Breakdown of Bradykinin and Its Metabolites as a Possible Cause for Pulmonary Edema in COVID-19 Infection. Semin Thromb Hemost. 46(7):835-37. (2020).
10. van de Veerdonk F L, Kouijzer I J E, de Nooijer A H, van der Hoeven H G, Maas C, Netea M G, Bruggemann R J M. Outcomes Associated With Use of a Kinin B2 Receptor Antagonist Among Patients With COVID-19. JAMA Netw Open. 3(8):e2017708 (2020).
11. Gangnus T, Burckhardt B B. Sensitive mass spectrometric determination of kinin-kallikrein system peptides in light of COVID-19. Sci Rep. 11(1):3061 (2020).
12. Benjamini, Yoav, and Yosef Hochberg. Controlling the False Discovery Rate: A Practical and Powerful Approach to Multiple Testing. Journal of the Royal Statistical Society. Series B (Methodological) 57(1):289-300 (1995).
13. Reddy R, Asante I, Liu S, Parikh P, Liebler J, Borok Z, Rodgers K, Baydur A, Louie S G. Circulating angiotensin peptides levels in Acute Respiratory Distress Syndrome correlate with clinical outcomes: A pilot study. PLoS One. 14(3):e0213096 (2019).

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A method of detecting an acute inflammatory response, comprising quantifying a peptide within the kallikrein-kinin system (KKS), the renin-angiotensin system (RAS), or a combination thereof, in a bodily fluid sample, wherein the level of the peptide in the bodily fluid sample is indicative of an acute inflammatory response, or a lack thereof.

2. A method of preparing a bodily fluid sample that is useful for detecting an acute inflammatory response, comprising:
a) obtaining or having obtained a bodily fluid sample;
b) adding a protease inhibitor, a control peptide, a retention-time standard peptide, or a combination thereof to the bodily fluid sample to prepare a sample that is useful for detecting an acute inflammatory response; and
c) quantifying a peptide within the kallikrein-kinin system (KKS), the renin-angiotensin system (RAS), or a combination thereof, in the sample prepared in step b).

3. The method of claim 2, further comprising enriching the peptide within the kallikrein-kinin system (KKS), the renin-angiotensin system (RAS), or the combination thereof, in the sample prior to quantifying in step c).

4. The method of any one of claims 1-3, wherein the bodily fluid sample is from a patient having a Severe Acute Respiratory Syndrome-related Coronavirus 2 (SARS-CoV2) infection.

5. A method of predicting a likelihood of developing an acute inflammatory response in a patient having a viral infection, comprising quantifying a peptide within the kallikrein-kinin system (KKS), the renin-angiotensin system (RAS), or a combination thereof, in a bodily fluid sample from the patient, wherein the level of the peptide in the bodily fluid sample is indicative of the likelihood of developing an acute inflammatory response in the patient.

6. A method of classifying a patient having a viral infection based on a predicted likelihood of developing an acute inflammatory response, comprising:
a) quantifying a peptide within the kallikrein-kinin system (KKS), the renin-angiotensin system (RAS), or a combination thereof, in a bodily fluid sample from the patient;
b) predicting the likelihood of developing an acute inflammatory response based on the level of the peptide in the bodily fluid sample; and
c) classifying the patient based on the predicted likelihood.

7. A method of treating a patient having a viral infection, comprising:
a) quantifying a peptide within the kallikrein-kinin system (KKS), the renin-angiotensin system (RAS), or a combination thereof, in a bodily fluid sample of the patient;
b) identifying the patient as being likely to develop an acute inflammatory response based on the level of the peptide in the bodily fluid sample; and
c) administering a therapy to the patient to inhibit acute inflammation.

8. A method of monitoring progression of an acute inflammatory response in a patient having a viral infection, comprising:
a) quantifying a peptide within the kallikrein-kinin system (KKS), the renin-angiotensin system (RAS), or a combination thereof, in a bodily fluid sample from the patient at a first time point;
b) repeating step a) at a second time point;
c) comparing the levels of the peptide in the bodily fluid sample at the first the second time points; and
d) determining the progression of the acute respiratory distress syndrome in the patient based on a change, or a lack thereof, in the levels of the peptide in the bodily fluid sample at the first and second time points.

9. The method of claim 8, wherein a therapy is administered to the patient between the first the second time points.

10. A method of stratifying a set of patients having a viral infection, comprising:
a) quantifying a peptide within the kallikrein-kinin system (KKS), the renin-angiotensin system (RAS), or a combination thereof, in bodily fluid samples from individual patients in the set, wherein the level of the peptide in a bodily fluid sample is indicative of the likelihood of developing an acute inflammatory response in a patient; and
b) stratifying the set of patients for treatment according to the individual patients' levels of the peptide in the bodily fluid samples.

11. A method of ranking an urgency for treatment in a set of patients having a viral infection, comprising:
a) quantifying a peptide within the kallikrein-kinin system (KKS), the renin-angiotensin system (RAS), or a combination thereof, in bodily fluid samples from individual patients in the set, wherein the level of the peptide in a bodily fluid sample is indicative of the likelihood of developing an acute inflammatory response in a patient; and
b) ranking the urgency for treating an acute inflammatory response in the set of patients according to the individual patients' levels of the peptide in the bodily fluid samples.

12. The method of any one of claims 1 and 4-11, further comprising adding a protease inhibitor, a control peptide, a retention-time standard peptide, or a combination thereof to the bodily fluid sample.

13. A method of preparing a bodily fluid sample useful for predicting an acute inflammatory response in a patient having a viral infection, comprising:
a) obtaining or having obtained the bodily fluid sample from the patient;
b) adding a protease inhibitor, a control peptide, a retention-time standard peptide, or a combination thereof to the bodily fluid sample to prepare a sample that is useful for predicting an acute inflammatory response in a patient; and
c) quantifying a peptide within the kallikrein-kinin system (KKS), the renin-angiotensin system (RAS), or a combination thereof, in the sample prepared in step b).

14. A method of processing a bodily fluid sample for detection of a peptide indicative of a likelihood of developing an acute inflammatory response, comprising:
a) receiving a bodily fluid sample from a customer, wherein the bodily fluid sample was obtained from a patient having or suspected of having a viral infection;
b) adding a protease inhibitor, a control peptide, a retention-time standard peptide, or a combination thereof to the bodily fluid sample to prepare a sample useful for detecting an acute inflammatory response in a patient;
c) quantifying a peptide within the kallikrein-kinin system (KKS), the renin-angiotensin system (RAS), or a combination thereof, in the sample prepared in step b);
d) generating a report based on levels of the peptide in the bodily fluid sample; and
e) delivering the report to the customer.

15. A method of providing information regarding a patient's likelihood of developing an acute inflammatory response, comprising:
a) receiving a bodily fluid sample from a customer, wherein the bodily fluid sample was obtained from a patient having or suspected of having a viral infection;
b) adding a protease inhibitor, a control peptide, a retention-time standard peptide, or a combination thereof to the bodily fluid sample to prepare a sample useful for detecting an acute inflammatory response in a patient;
c) quantifying a peptide within the kallikrein-kinin system (KKS), the renin-angiotensin system (RAS), or a combination thereof, in the sample prepared in step b);
d) generating a report based on levels of the peptide in the bodily fluid sample; and
e) delivering the report to the customer.

16. The method claim 14 or 15, wherein the customer is a hospital, a doctor's office, a medical research lab or facility, a government agency, or a combination thereof.

17. The method of any one of claims 12-16, further comprising enriching the peptide within the kallikrein-kinin system (KKS), the renin-angiotensin system (RAS), or the combination thereof, in the sample.

18. The method of any one of claims 12-17, wherein the protease inhibitor targets Cathepsin B, ACE2, Thimit oligopeptidase, MMP8, Neprilysin, ECE-1, ECE-2, Neprilysin-2, Chymase, Neutrophil elastase, tissue kallikrein, plasma kallikrein, aminopeptidase P, aminopeptidase M or a combination thereof.

19. The method of any one of claims 12-17, wherein the protease inhibitor is selected from the group consisting of: leupeptin, E64d, antipain, DX600, MLN-4760, EDTA, RXP03, phosphoramidon, candoxatril, SM-19712, S136492, Chymostatin, alpha antichymotrypsin, Sivelestat, an ACE-inhibitor and combinations thereof.

20. The method of any one of claims 12-19, wherein the control peptide is a stable-isotope labeled analog of the one or more peptides within the kallikrein-kinin system (KKS), the renin-angiotensin system (RAS), or a combination thereof.

21. The method of any one of claims 12-20, wherein the retention-time standard peptide is selected from the group consisting of: LGGNETQVR (SEQ ID NO:3), AGGSSEPVTGLADK (SEQ ID NO:4), VEATFGVDESANK (SEQ ID NO:5), YILAGVESNK (SEQ ID NO:6), TPVISGGPYYER (SEQ ID NO:7), TPVITGAPYYER (SEQ ID NO:8), GDLDAASYYAPVR (SEQ ID NO:39), DAVTPADFSEWSK (SEQ ID NO:10), TGFIIDPGGVIR (SEQ ID NO:11), GTFIIDPAAIVR (SEQ ID NO:12), FLLQFGAQGSPLFK (SEQ ID NO:13) and combinations thereof.

22. The method of any one of the preceding claims, wherein the peptide within the kallikrein-kinin system (KKS), the renin-angiotensin system (RAS), or the combination thereof, in the bodily fluid sample is quantified using a mass-spectrometry-based assay, an antibody-based assay, or both.

23. The method claim 22, wherein the peptide within the kallikrein-kinin system (KKS), the renin-angiotensin system (RAS), or the combination thereof, in the bodily fluid sample is quantified using a mass-spectrometry-based assay.

24. The method claim 23, wherein the mass-spectrometry-based assay is immuno-Matrix Assisted Laser Desorption/Ionization (iMALDI).

25. The method claim 24, wherein the iMALDI is a multiplexed iMALDI.

26. The method of any one of claims 5-25, wherein the patient is infected with a SARS-CoV2.

27. The method of any one of claims 5-26, wherein the patient has been diagnosed with COVID-19.

28. The method of any one of the preceding claims, wherein the peptide within the kallikrein-kinin system (KKS) is selected from the group consisting of Kallidin (K-BK[1-9]), Bradykinin, [Des-Arg9]-Bradykinin, Lys-[Des-Arg9]-Bradykinin, RPPGF SP (SEQ ID NO:14), KRPPGFSP (SEQ ID NO:15), RPPGF (SEQ ID NO:22), KRPPGF (SEQ ID NO:23) and combinations thereof.

29. The method of claim 28, wherein the peptide within the kallikrein-kinin system (KKS) is Kallidin (K-BK[1-9]).

30. The method of any one of the preceding claims, wherein the bodily fluid sample is a plasma sample.

31. The method of claim 30, wherein an above-threshold level of Kallidin in plasma is indicative of a higher likelihood for the patient to develop an acute inflammatory response.

32. The method of any one of claims 28-31, further comprising quantifying a peptide within the renin-angiotensin system (RAS) in a saliva sample.

33. The method of claim 32, wherein the peptide within the renin-angiotensin system (RAS) is selected from the group consisting of Angiotensin I (Ang I), Angiotensin III (Ang[2-8]), Angiotensinogen (ANGT), Angiotensin [1-9] (Ang-[1-9]), Angiotensin II (Ang II), Angiotensin-[1-7] (Ang-[1-7]), and combinations thereof.

34. The method of claim 32, wherein the peptide within the renin-angiotensin system (RAS) is Angiotensin I (Ang I), Angiotensin III (Ang[2-8]), or both.

35. The method of claim 32, wherein:
a) a below-threshold level of Angiotensin I (Ang I) in saliva;
b) a below-threshold level of Angiotensin III (Ang[2-8]) in saliva; or
c) both a) and b),
provides further indication of a higher likelihood for the patient to develop an acute inflammatory response.

36. The method of any one of claims 31-35, further comprising administering a therapy to inhibit acute inflammation in a patient identified as having a higher likelihood of developing an acute inflammatory response.

37. The method of claim 31-36, wherein the acute respiratory distress syndrome is a severe acute respiratory distress syndrome.

38. The method of claim 7, 36 or 37, wherein the therapy comprises a therapeutically effective amount of an agent that inhibits AT1R, an agent that blocks ANG II production, or a combination thereof.

39. The method of claim 38, wherein the agent that inhibits AT1R includes a sartan, an Angiotensin II receptor blocker (ARB), or a combination thereof.

40. The method of claim 38, wherein the agent that blocks ANG II production includes an Angiotensin-converting-enzyme inhibitor (ACE inhibitor), a renin inhibitor, or a combination thereof.

41. The method of claim 7, 36 or 37, wherein the therapy comprises an agent selected from the group consisting of: an ACE inhibitor, an Angiotensin II type-1 receptor blocker, an Angiotensin II type-2 receptor agonist, a MAS receptor agonist, an ACE2 activator, an agent that blocks binding interface between SARS-CoV2 and ACE2, a soluble ACE2, an agent that blocks ANG II production, and combinations thereof.

42. The method of claim 7, 36 or 37, wherein the therapy comprises an agent that increases:
a) expression of Alamandine receptor, AT1R, AT2R, AT4, Mas, MrgD receptor or a combination thereof;
b) a function of Alamandine receptor, AT1R, AT2R, AT4, Mas, MrgD receptor or a combination thereof; or
c) both a) and b).

43. The method of claim 42, wherein the agent is selected from the group consisting of Ang II, C-reactive protein, an Ang-[1-7] analog, or a combination thereof.

44. The method of claim 43, wherein the Ang-[1-7] analog comprises cyclic Ang[1-7] or NorLeu3 Ang[1-7], or a combination thereof.

45. The method of claim 7, 36 or 37, wherein the therapy comprises an agent selected from the group consisting of: Captopril, candesartan, Compound 21 (C21), AVE 0991, rhACE2, rhACE2, Anti-ACE2 antibody, and combinations thereof.

46. The method of claim 7, 36 or 37, wherein the therapy comprises a therapeutically effective amount of:
a) an agent that inhibits B1R, B2R, or both B1R and B2R;
b) an agent that blocks tissue kallikrein activity, plasma kallikrein activity, or both;
c) an agent that blocks the production of kallidin, bradykinin, low molecular weight kinin, high molecular weight kinin, kininogen, BK[1-8] or K-BK[1-8], or a combination thereof; or
a combination of a)-c).

47. The method of claim 46, wherein the agent that inhibits B2R is a B2-receptor antagonist.

48. The method of claim 47, wherein the B2-receptor antagonist is icatibant.

49. The method of any one of claims 5-48, further comprising admitting a patient identified as having a higher likelihood of developing a severe acute respiratory distress syndrome for in-hospital treatment.

50. The method of claim 49, further comprising triaging the patient based on the likelihood for the patient to develop an acute inflammatory response or a severe acute respiratory distress syndrome.

51. The method of any one of claims 5-50, further comprising analyzing a genetic risk factor associated with a likelihood of developing the acute inflammatory response in the patient.

52. The method of claim 51, wherein the genetic risk factor is associated with an increased likelihood of developing the acute inflammatory response.

53. The method of claim 51 or 52, wherein the genetic risk factor is a gene variant associated with an increased severity of the acute inflammatory response.

54. The method of any one of claims 51-53, wherein the gene variant is a variant of a gene selected from the group consisting of ACE, ACE2, AGT, Apolipoprotein E (Apo E), AT1R, aminopeptidase P (APP), kallikrein, Mannose-Biding Lectin, CD147, CCL2, Interleukin-12, and a human leukocyte antigen (HLA) class II gene, a HLA class III gene, and combinations thereof.


SEQUENCE LISTING (24 sequences)

SEQ ID NO: 1 - protein, 10 aa, Homo sapiens
DRVYIHPFHL

SEQ ID NO: 2 - protein, 8 aa, Homo sapiens
DRVYIHPF

SEQ ID NO: 3 - protein, 9 aa, Artificial Sequence
  retention-time standard peptide
LGGNETQVR

SEQ ID NO: 4 - protein, 14 aa, Artificial Sequence
  retention-time standard peptide
AGGSSEPVTGLADK

SEQ ID NO: 5 - protein, 13 aa, Artificial Sequence
  retention-time standard peptide
VEATFGVDESANK

SEQ ID NO: 6 - protein, 10 aa, Artificial Sequence
  retention-time standard peptide
YILAGVESNK

SEQ ID NO: 7 - protein, 12 aa, Artificial Sequence
  retention-time standard peptide
TPVISGGPYYER

SEQ ID NO: 8 - protein, 12 aa, Artificial Sequence
  retention-time standard peptide
TPVITGAPYYER

SEQ ID NO: 9 - protein, 13 aa, Artificial Sequence
  retention-time standard peptide
GDLDAASYYAPVR

SEQ ID NO: 10 - protein, 13 aa, Artificial Sequence
  retention-time standard peptide
DAVTPADFSEWSK

SEQ ID NO: 11 - protein, 12 aa, Artificial Sequence
  retention-time standard peptide
TGFIIDPGGVIR

SEQ ID NO: 12 - protein, 12 aa, Artificial Sequence
  retention-time standard peptide
GTFIIDPAAIVR

SEQ ID NO: 13 - protein, 14 aa, Artificial Sequence
  retention-time standard peptide
FLLQFGAQGSPLFK

SEQ ID NO: 14 - protein, 7 aa, Homo sapiens
RPPGFSP

SEQ ID NO: 15 - protein, 8 aa, Homo sapiens
KRPPGFSP

SEQ ID NO: 16 - protein, 9 aa, Homo sapiens
DRVYIHPFH

SEQ ID NO: 17 - protein, 7 aa, Homo sapiens
DRVYIHP

SEQ ID NO: 18 - protein, 7 aa, Homo sapiens
RVYIHPF

SEQ ID NO: 19 - protein, 10 aa, Homo sapiens
KRPPGFSPFR

SEQ ID NO: 20 - protein, 9 aa, Homo sapiens
RPPGFSPFR

SEQ ID NO: 21 - protein, 8 aa, Homo sapiens
RPPGFSPF

SEQ ID NO: 22 - protein, 5 aa, Homo sapiens
RPPGF

SEQ ID NO: 23 - protein, 6 aa, Homo sapiens
KRPPGF

SEQ ID NO: 24 - protein, 485 aa, Homo sapiens
MRKRAPQSEMAPAGVSLRATILCLLAWAGLAAGDRVYIHPFHLVIHNESTCEQLAKANAGKPKDPTFIPAPIQAKTSPVDEKALQDQLVLVAAKLDTEDKLRAAMVGMLANFLGFRIYGMHSELWGVVHGATVLSPTAVFGTLASLYLGALDHTADRLQAILGVPWKDKNCTSRLDAHKVLSALQAVQGLLVAQGRADSQAQLLLSTVVGVFTAPGLHLKQPFVQGLALYTPVVLPRSLDFTELDVAAEKIDRFMQAVTGWKTGCSLMGASVDSTLAFNTYVHFQGKMKGFSLLAEPQEFWVDNSTSVSVPMLSGMGTFQHWSDIQDNFSVTQVPFTESACLLLIQPHYASDLDKVEGLTFQQNSLNWMKKLSPRTIHLTMPQLVLQGSYDLQDLLAQAELPAILHTELNLQKLSNDRIRVGEVLNSIFFELEADEREPTESTQQLNKPEVLEVTLNRPFLFAVYDQSATALHFLGRVANPLSTA